(19) United States
(12) Patent Application Publication
(10) Pub. No.: US 2011/0003279 A1
(43) Pub. Date: Jan. 6, 2011

(54) Monitoring devices and processes based on transformation, destruction and conversion of nanostructures

(76) Inventor: Gordhanbhai Nathalal Patel, Somerset, NJ (US)

Correspondence Address: Barbara V. Maurer, 5 Haven Hill Rise, Flemington, NJ, 08822, US

(21) Appl. No.: 12/879,688

(22) Filed: Sep. 10, 2010

Related U.S. Application Data
(63) Continuation-in-part of application No. 12/478,232, filed on Jun. 4, 2009.
(60) Provisional application No. 61/276,349, filed on Sep. 11, 2009.

(51) Int. Cl. C12Q 1/70; C12Q 1/02; G01N 31/00; G01T 1/00; G01K 3/00; G01J 4/00

(52) U.S. Cl. 435/5; 435/29; 436/1; 250/336.1; 374/104; 356/369; 977/700; 977/774; 977/773; 977/902; 374/E03.001; 977/953

(57) ABSTRACT

A large number of properties of nanostructures depend on their size, shape and many other parameters. As the size of a nanostructure decreases, there is a rapid change in many properties. When the nanostructure is completely destroyed, those properties essentially disappear. Systems based on changes in properties of nanostructures due to the destruction of nanostructures are proposed. The systems can be used for monitoring the total exposure to organic, inorganic, organometallic and biological compounds and agents using analytical methods.

### CROSS-REFERENCES TO RELATED APPLICATIONS

This application is a CIP of U.S. patent application Ser. No. 12/478,232 and U.S. provisional patent applications cited therein, incorporated herein by reference in their entireties.

This application claims priority to U.S. Provisional Patent Applications No. 61/132,799, filed Dec. 15, 2008; 61/162,539, filed Mar. 23, 2009; 61/215,982 filed May 12, 2009; and 61/276,349 filed Sep. 11, 2009.

### FIELD OF INVENTION

This invention relates to devices and associated processes based on physical, chemical and biological destruction of nanostructures. This invention also relates to monitoring the total exposure to organic, inorganic, organometallic and biological compounds and agents using unstable, reactive or destructible nanostructures using analytical methods.

### BACKGROUND OF THE INVENTION

U.S. patent application Ser. No. 12/478,232 discloses certain formulations and devices based on the etching of a thin (e.g., 10-100 nm) layer of a metal and fine (1-50 microns) particles (destruction of a nano-structure) including some methods for monitoring and measuring concentrations of chemical and biological agents.

A nanostructure is an object made from an atom or molecule to a microscopic size. Except a quantum dot, nanostructures have at least one dimension usually between 1 and 100 nanometers and usually a narrow size distribution. A lightly metallized plastic film, has one dimension on the nanoscale, i.e., only the thickness of the metal layer is between 0.1 and 100 nm. Nanowires are one dimensional, nanotubes have two dimensions on the nanoscale, i.e., the diameter of the tube is between 0.1 and 100 nm; its length could be much greater. Finally, spherical nanoparticles have three dimensions on the nanoscale, i.e., the particle is between 0.1 and 100 nm in each spatial dimension.

Materials reduced to the nanoscale can show very different properties compared to what they exhibit on a macro scale, enabling unique applications. For instance, opaque substances become transparent (copper), inert materials attain catalytic properties (platinum), stable materials turn combustible (aluminum), solids turn into liquids at room temperature (gold) and insulators become conductors (silicon). Materials, such as gold, which is chemically inert at normal scales, can serve as a potent chemical catalyst at nanoscales. Much of the interest in nanotechnology stems from the unique quantum and surface phenomena that a matter exhibits at the nanoscale.

Nanostructures often have unusual visual properties because they are small enough to confine their electrons and produce quantum effects. For example gold nanoparticles appear deep red to black in solution. As there is a gradual transition from normal nano (e.g., 10 nm) to a nanometer and lower, there will be several other changes in properties at an atomic level and hence can undergo a variety of changes.

Nanotechnology is used in many commercial products and processes. Nanomaterials are used to add strength to composite materials used to make lightweight tennis rackets, baseball bats, and bicycles. Nanostructured catalysts are used to make chemical manufacturing processes more efficient, saving energy and reducing the waste products. A few pharmaceutical products have been reformulated with nanosized particles to improve their absorption and make them easier to administer. Opticians apply nanocoatings to eyeglasses to make them easier to keep clean and harder to scratch. Nanomaterials are applied as coatings on fabrics to make clothing stain resistant and easy to care for. Nanoceramics are used in some dental implants, or to fill holes in bones after removing a bone tumor, because their mechanical and chemical properties can be tuned to match those of the surrounding tissue. Many electronic devices manufactured in the last decade use some nanomaterials. Nanotechnology is used much more extensively to build new transistor structures and interconnects for the fastest, most advanced computing chips. Characterization of nano structures is done by using a variety of different techniques, such as electron microscopy, atomic force microscopy (AFM), dynamic light scattering, X-ray photoelectron spectroscopy, powder X-ray diffractometry, fourier transform IR, matrix-assisted laser desorption, time-of-flight mass spectroscopy and UV visible spectroscopy.

A number of devices and products are reported based on nanostructures. Those devices and products are based on stable nanostructures.

Nanostructures are intrinsically less stable than their counter microstructures. There are many reports on making nanostructures, their unique properties and products made from them, for example, A. Henglein., Chem. Rev., 89 (1989) 1861; M. B. Mohamed, C. Burda, and M. A. El-Sayed, Nanolett., 1 (2001) 589; J. H. Fendler, Chem. Mater., 8 (1996) 1616; C. R. Henry, Surf Sci. Rep. 31, 231 (1998). There are no reports, however, on devices and processes based on destruction of nanostructures.

### SUMMARY OF THE INVENTION

Thus it is an object of the invention to use this phenomenon to create a variety of devices, products and processes. It is also an object of the present inventions to develop devices, products and processes based on (1) destruction, including reduction in size of nanostructures, (2) higher reactivity of nanostructures, (3) rapid change in properties when size of nanostructures is changed, (4) using unstable nanostructures and alike.

Thus, this invention relates to an indicating system which comprises a nanostructure; and a means to measure the change in properties of the nanostructure as it is destroyed. In the indicating system the destruction is due to one or more of: melting, fusion, dissolution, swelling, drying, etching, coagulation, conversion, transformation, crystallization, formation of defects, decomposition, reaction, diffusion, complex or adduction formation, transformation, phase, reactivity, state, size, shape, nature of doping, magnetism, porosity, permeability degradation, decay, corrosion, decomposition, disintegration, deterioration, de-metallization, coalescence, adsorption, desorption, melting, crystallization, phase change, electronic or nuclear structure, magnetism, and optical properties. The nanostructure is typically less than about 1,000 nm in at least one dimension.

The nanostructure is comprised of one or more structures selected from the group of nanoantenna, nanoballs, nanobelts, nanobipods, nanocapsules, nanocluster, nanocrystals, branched nanocrystals, nanodendrimers, nanodots, nanofilms, nanofibers, nanoflakes/sheets, nanofluids, nanolayers, nanoparticles, nanorods, nanospheres, nanosprings, nanotatrapods, branched tetrapods, nanotripods, nanotubes, nanowires, plasmon, quantum dots, and quantum wells. The nanostructure is generally a reactive or unstable organic, inorganic, organo-metallic or a biological material and can also be made from a metal, such as for example, copper, zinc, magnesium, aluminum, gold, silver silicon, or their alloys.

The indicating system of the invention is based on the destruction of a nanostructure wherein the nanostructure is destroyed by an analyte or activator. The analyte can be selected from a chemical or biological agent. In one embodiment, the chemical agent is a toxic or hazardous chemical. In another embodiment, the biological agent is a virus or a bacterium.

In yet another embodiment, the analyte is energy, electromagnetic radiation, pressure, or magnetism.

The invention also relates to a process of measuring change in a property of a nanostructure during its destruction, as described more fully below.

Another embodiment relates to a process of changing the performance of an indicating nanostructure device which comprises changing a non-linear performance of the indicating device to a linear performance by increasing the size distribution of the nanostructures in the indicating system.

In one embodiment of the invention, the indicating system is designed for use in monitoring total exposure to organic, inorganic, organometallic and biological compounds and agents or analytes using analytical methods.

In another embodiment of the invention, the indicating system is designed for monitoring time, time-temperature, thaw, freeze, humidity, ionizing radiation, temperature, microwave, sterilization, chemicals, biological or chemical agents, wherein the sterilization is done with steam, ethylene oxide, plasma, formaldehyde, dry heat, hydrogen peroxide or peracetic acid.

In yet another embodiment, the indicating system of the invention is a radiation dosimeter, such as a capacitor.

In the indicating system of the invention the nanostructure can be an electrode, such as an organic or inorganic conductor, semiconductor or metal electrode.

In some aspects of the invention, the nanostructure is protected by a coating or stabilizing material which is a precursor, activator or transparent conductor. A preferred precursor is a halo-compound.

In one aspect of the invention the destruction of the nanostructure is determined an analytical method, including an electroanalytical method, such as, for example ellipsometry.

A main objective of this invention is to provide a system of indicating devices for monitoring materials and processes such as time, temperature, time-temperature, thaw, freeze, humidity, ionizing radiation, microwave, sterilization (including steam, ethylene oxide, plasma, formaldehyde, dry heat, hydrogen peroxide and peracetic acid), chemicals, biological and chemical agents, and electronic devices, such as RFID (radio frequency identification device) and EAS (Electronic article surveillance), printed electrodes and alike based on destruction of nanostructures.

In one aspect of the invention there are provided reactive/destructible nano sensor systems for monitoring a variety of processes such as time, temperature, time-temperature, thaw, freeze, humidity, ionizing radiation, microwave, sterilization (including steam, ethylene oxide, plasma, formaldehyde, dry heat, hydrogen peroxide and peracetic acid), chemicals, biological and chemical agents, and electronic devices, such as RFID and EAS, printed electrodes and alike based on the destruction of nanostructures.

Also provided are sensors and similar devices made from destructible nanostructures that convert physical, biological or chemical input into an electrical or optical signal. The signal measures and transforms into digital format which can then be processed and analyzed efficiently by computers. The information can be used by either a person or an intelligent device monitoring the activity to make decisions that maintain or change a course of action.

Additionally there is provided a system/device that measures a substantially irreversible change in physical or chemical properties of nanostructure and provides a signal which can be read by an observer or by an instrument.

In aspects of the invention related to analytes, there is are preferred nanostructures which are unstable and reactive to analytes or activators.

Also provided is a process of monitoring analytes composed of certain ions and metals, such as those of toxic elements, such as lead (Pb), mercury (Hg), arsenic (As), chromium (Cr), cadmium (Cd), barium (Ba), silver (Ag), and selenium (Se) pose significant health risks when present in water supplies with a destroyable nanostructure.

It is an additional object of the invention to provide methods for monitoring agents using a sensor having a destroyable nanostructure. The methods include noncontact and nondestructive methods, such as optical technique, spectroscopic and ellipsometry.

Also provided are devices and methods for determination of change in properties due of a destruction of a nanostructure with analytic equipment or technique.

Provided also are methods and devices for the creation of nanostructures and quantum devices, such as nanoantenna, nanowires, nanodots and quantum dots, e.g., by the etching or dissolution of metals and their alloys, semi-metals, semi-conductors and doped organic and inorganic materials including semiconducting and conducting materials, such as conducting polymers.

Provided are methods for monitoring analytes, such as chemical and biological agents using a destructible nanostructure, e.g., a very thin layer or nano sized particles of electrically conductive materials, such as metals, alloys and/or an oxide layer on them. They also include use of the assembly as an electrode or electrochemical sensors.

Provided are methods of creating a wide range of devices, such as light emitting devices, capacitors, batteries, catalysts, electrochemical sensors, biosensors and materials, such as structural materials and the like by destruction of nanostructures or a layer or component having a nanostructure.

Provided are methods of making non-linear changes in properties of the indicating devices based on destruction of nanostructures to linear changes in properties.

Provided are methods of making non-linear changes in properties of the indicating devices based on destruction of nanostructures to linear changes in properties by using broad distribution of the nano structures.

Provided are nanostructures coated with at least one pre-cursor.

Provided are methods of coating nanostructures with a pre-cursor.

Provided are methods of monitoring changes in destructible nano-structures by visual and analytical methods.

Provided are indicating devices based on destructible nanostructures which are smaller than 5 nm.

Also provided are laminates of nanostructures which deteriorate upon exposure to an agent or analyte.

The reactive/destructible nanosensors of the invention can be dosimeters for monitoring radiation, ionizing radiation, X-ray, gamma ray, electrons, protons and neutrons. The dosimeters for monitoring ionizing radiation monitor change in electrical resistance, capacitance, optical properties and thickness, using, for example, LED, capacitor, diffraction grating, diode and photocell containing reactive/destructible nanosensors.

Also provided are methods for monitoring ionizing radiation using reactive/destructible nanosensors as dosimeters.

In addition there is provided a destructible layer of nanostructures comprising at least one nanostructure, wherein the nanostructure layer is optically transparent, semitransparent, semiconductive and/or electrically conductive.

Also provided is a machine, apparatus, equipment for determination of effect of an activator on a destructible nanostructure including indicator/electrode/conductor connected to a power source.

Provided is a machine, apparatus, equipment wherein effect of activator on a destructible nanostructure including indicator/electrode/conductor is determined by determining change in electromagnetic properties.

In another aspect of the invention there is provided a system for simultaneously monitoring multiple analytes in a sample using a destroyable quantum dot (QD).

Also provided is an indicating system for simultaneously monitoring multiple analytes in a sample, comprising: a first irreversibly reactive QD that reacts to a first analyte; a second reactive QD that reacts to a second analyte; and so on. There may be one or more quencher, for quenching the emissions of QDs.

Also provided is an indicating system having more than one destructible nanostructure including quantum dots that comprise at least one member selected from the group consisting of CdS, CdSe, CdTe, ZnS, ZnSe, ZnTe, Pin, PbSe, CdZnSe and a destroyable nanostructure.

Also provided are methods of destruction, including methods of making nanostructures from materials which are susceptible to analytes and a technique for creating destroyable nanostructures by etching larger nanostructures.

Diode and electronic devices of the invention include an apparatus comprising a destroyable Schottky diode made from inorganic and organic semi-conductors having one or more destroyable components. Such diode can be comprised of a silicon substrate; an ultrathin destroyable metal film located on a portion of said silicon substrate; said ultrathin metal film and said silicon substrate together forming a Schottky barrier having the current-voltage characteristics of a diode thereby enabling detection of a surface adsorbate/reaction on said ultrathin destroyable metal film; wherein the presence of said surface adsorbate creates a measurable current resulting from production of electrons or holes having sufficient energy to transverse said ultrathin metal film and cross said Schottky barrier; an oxide layer formed on said silicon substrate and having an inclination formed therein; and at least one zero force electrical contact including a metalized contact electrically connected to said ultrathin destroyable metal film; said metalized contact being deposited on said oxide layer and wherein said ultrathin metal includes a portion deposited on top of said inclination in the oxide layer before being connected to the metalized contact.

Also provided is destroyable capacitor having two reactive metal layers having thickness in nanometers and a dielectric layer which has capability of producing an activator when subjected to an analyte, such as electromagnetic radiation (e.g., X-ray) and magnetism.

Still another object is to provide a partially demetallized semiconductive metal susceptor for microwave indicator wherein the heat produced in different areas can be precisely controlled and the various areas producing different amounts of heat can be given any desired shape.

### BRIEF DESCRIPTION OF THE FIGURES

FIG. 1 shows a schematic presentation of changes in some properties of nano materials with the size of nanostructures.

FIG. 2 shows a schematic presentation of a change in a property, such as transparency or electrical resistance with the thickness of metallized (aluminized) plastic film or aluminum particles during an etching process.

FIG. 3 shows a schematic presentation of a change in (disappearance or absence of) a property upon the destruction of a nanostructure.

FIG. 4 shows a schematic presentation of the creation of a nanowall (b), nanorod (c), thin nanofilm (d), nanowire/fiber (e) and quantumdot/nanodot (f) by selective etching of a nanofilm (a) on a substrate (e.g., a metallized plastic film).

FIG. 5 shows a schematic presentation of a dosimeter sensor device made from nanowires (1) and two electrodes with terminals (2) on a substrate (3). The device may have coating of a precursor (not shown).

FIG. 6 shows a schematic presentation of a change in property with the size of a nanostructure having a narrow size distribution (top curve) and with a broad random distribution (lower line).

FIG. 7 shows a schematic cross sectional presentation of a dosimeter sensor having a layer for the transport/injection of an electron (2) between cathode (1) and an electroactive layer (3), and a layer for transport of holes (4) between the electroactive layer and anode (5).

FIG. 8 shows a schematic cross sectional presentation of different layers of a dosimeter sensor device made from different convertible semiconductor layers, insulator/dielectric layers and conductors.

FIG. 9 shows a schematic cross sectional presentation of a de-activatable magnetic EAS system.

FIG. 10 shows a schematic cross sectional presentation of a pyro or piezo electric de-activatable transducer. The conductive layer can be indium tin oxide (ITO).

FIG. 11 shows a flow chart of an apparatus having a nano diffraction grating as a sensor. The grating sensor can be an optical fiber.

FIG. 12 shows a schematic cross sectional presentation of a dosimeter light emitting diode (LED) having a convertible phosphor layer before (a) medium (b) and high (b) dose of an analyte, such as X-ray.

FIG. 13 shows a schematic cross sectional presentation of a dosimeter photocell having a susceptible photo absorbing layer before (a) and after (b) exposure to an analyte, such as X-ray.

FIG. 14 shows a schematic presentation of a dosimeter/detector diode having at least one susceptible component.

FIG. 15 shows a schematic presentation of some representative examples of different types of susceptible nano antennas or sensors.

FIG. 16 shows a schematic presentation of dosimeter nano antennas/sensors made from different susceptible materials or coated with different precursors for monitoring different agents.

FIG. 17 shows a schematic presentation of susceptible nano antennas/sensors coated with different precursors for monitoring different agents.

FIG. 18 shows a schematic presentation of a number of destructible nano antennas/sensors connected in a series.

FIG. 19 shows a schematic presentation of a number of destructible nano antennas/sensors connected in a series and coated with different precursors for monitoring different agents. Each antenna/sensor can be made individually addressable.

FIG. 20 shows a schematic presentation of a dosimeter device for measurement of change in parameters, such as resistance of a conductive or semiconductive nano layer upon exposure to analytes.

FIG. 21 shows a schematic presentation of a radiation dosimeter device (capacitor) and apparatus for measurement of change in more than one parameter, such as resistance of a susceptible nano thin electrode and capacitance of the device upon exposure to high energy radiation, such as X-ray. An example is described in Example 1

FIG. 22 shows a schematic presentation of a radiation dosimeter/sensor (rolled capacitor) having two alternating layers of a susceptible nano thin electrode and a dielectric layer containing a precursor.

FIG. 23 shows a schematic presentation of a radiation dosimeter/sensor (rolled capacitor) having a dielectric layer containing a precursor between two layers of a susceptible nano thin electrode and a stable dielectric layer.

FIG. 24 shows a schematic presentation of a radiation dosimeter/sensor (rolled capacitor) having a dielectric layer containing a precursor between two layers of a non-destroyable thin electrode and a stable dielectric layer.

FIG. 25a shows a photograph of an experimental set up for determination of a change in resistance of a metallized PET film as a susceptible electrode having a thin coating of a precursor (a halocarbon) and then exposed to short wavelength UV light (blue glow).

FIG. 25b shows a photograph of the device of FIG. 25a after 2.5 hrs of UV exposure. Electrical resistance changed from 0.56 kilo Ohms (FIG. 25a) to 21.6 mega Ohms.

### DETAILED DESCRIPTION OF THE INVENTION

### Definitions

Unless defined otherwise, all technical and scientific terms used herein have the same meaning as commonly understood by one of ordinary skill in the art to which the invention pertains. The terminology used herein is for the purpose of describing particular embodiments only, and is not intended to be limiting.
Nanostructure: A “nanostructure” is a structure having at least one region or characteristic dimension with a dimension of less than about 1,000 nm, e.g., less than about 200 nm, less than about 100 nm, less than about 50 nm, less than about 10 nm or even less than a nanometer. Typically, the region or characteristic dimension will be along the smallest axis of the structure. Examples of such structures include nanoantenna, nanoballs (e.g., fullerenes or buckyballs), nanobelts, nanobipods, nanocapsules, nanocluster, nanocrystals, branched nanocrystals, nanodendrimers, nanodots, nanofilms, nanofibers, nanoflakes/sheets, nanofluids, nanolayers, nanoparticles, nanorods, nanospheres, nanosprings, nanotatrapods, branched tetrapods (e.g., dendrimers), nanotripods, nanotubes, nanowires, quantum dots, quantum wells, others listed herein and alike.

Nanostructures can be substantially homogeneous in material properties, or in certain embodiments can be heterogeneous (e.g. heterostructures). Nanostructures can be, e.g., substantially crystalline, substantially monocrystalline, polycrystalline, amorphous, or a combination thereof. The material of the nanostructures can be an organic, an organometallic, biological or inorganic (metallic, semiconducting and dielectric) chemical. Nanostructures can be natural or synthetic bionanostructures. Nanostructures can be functionalized or nonfunctionalized. They can be dispersed or coagulated. Nanostructures can be porous, hollow, solid, single or multilayered. Nanostructures herein include colloids, nanoemulsions, microemulsions and nano-sized liquid crystals, especially when an indicator, activator, precursor, additive or coating material is a liquid or semisolid.
Agents and analytes: An agent or analyte is one which has a capability of reacting or interacting with a nanostructure or a precursor for an activator and changes its property. An agent or analyte include non-materials/energy, such as electromagnetic radiation, pressure, magnetism, and materials, such as chemicals including organic, inorganic, organometallic and biological compounds. The term “analyte”, “biological analyte” or “chemical analyte” means a substance being measured in an analytical procedure. Non-material or energy type analytes, such as ionizing radiation, pressure, magnetism and alike which have substantial capability of passing through nanostructures and components of the devices made from a nanostructure and the other types include chemicals, biological agents which react/interact usually on the surface of a nanostructure. Time can also be an analyte, for example, in the case of time, time-temperature and other devices and processes.

The terms indicator, dosimeter, activator, precursor, binder, metallic, permeable and others used herein are as defined or described in Ser. No. 12/478,232.
Destroyable: The destroyable, susceptible and alike nanostructure means a nanostructure which undergoes one or more of a sufficiently, usually irreversible, noticeable or measurable change in physical, biological or chemical properties, including melting, fusion, dissolution, swelling, drying, etching, coagulation, conversion, transformation, crystallization, formation of defects, decomposition, reaction, diffusion, complex or adduction formation, transformation, phase, reactivity, state, size, shape, nature of doping (e.g., “p” and “n” type), magnetism, porosity, or permeability and alike.

A nano layer which substantially irreversibly degrades, decays, perishes, corrodes, rots, putrefies, decomposes, crumbles, disintegrates, deteriorates, destructs, becomes unstable or de-metallizes, undergoes some change in physical or chemical properties is also included in the definition of destructible nanostructure.

The destruction of a nanostructure can be due to many processes and materials including indicator, activator, additives and precursor. The destruction can be due to many physical, chemical and biological processes and materials. A chemical reaction, such as etching is just one of them. The destruction does not have to complete destruction of the nanostructure. It can be physical as well. Coalescence, adsorption, desorption, melting, crystallization, phase change, electronic or nuclear structure, magnetism, optical and alike.
Analytical instruments and methods: One or more methods/techniques commonly used in the analytical science, including those listed herein.
Sensor/dosimeter/indicator/indicating devices: A sensor means a device made from a nanostructure that responds to a stimulus, such as radiation, chemical or biological stimuli. The nanostructure can be destroyable. The term sensor, device, dosimeter, indicator, indicating devices etc are used interchangeably herein. Indicating devices of the present inventions, include devices for measuring for time, time-temperature, thaw, freeze, humidity, ionizing radiation, temperature, microwave, sterilization (including that with steam, ethylene oxide, plasma, formaldehyde, dry heat, hydrogen peroxide and peracetic acid), chemicals, biological and chemical agents, microwave and all other devices (e.g., printed circuit board, RFID and EAS), including those defined above and herein. An indicating device or indicating system also includes other formulations, devices and processes disclosed herein. We have also used the word integrator, integrating device, sensor, detector and monitor and monitoring devices interchangeably with indicating device and indicating system.

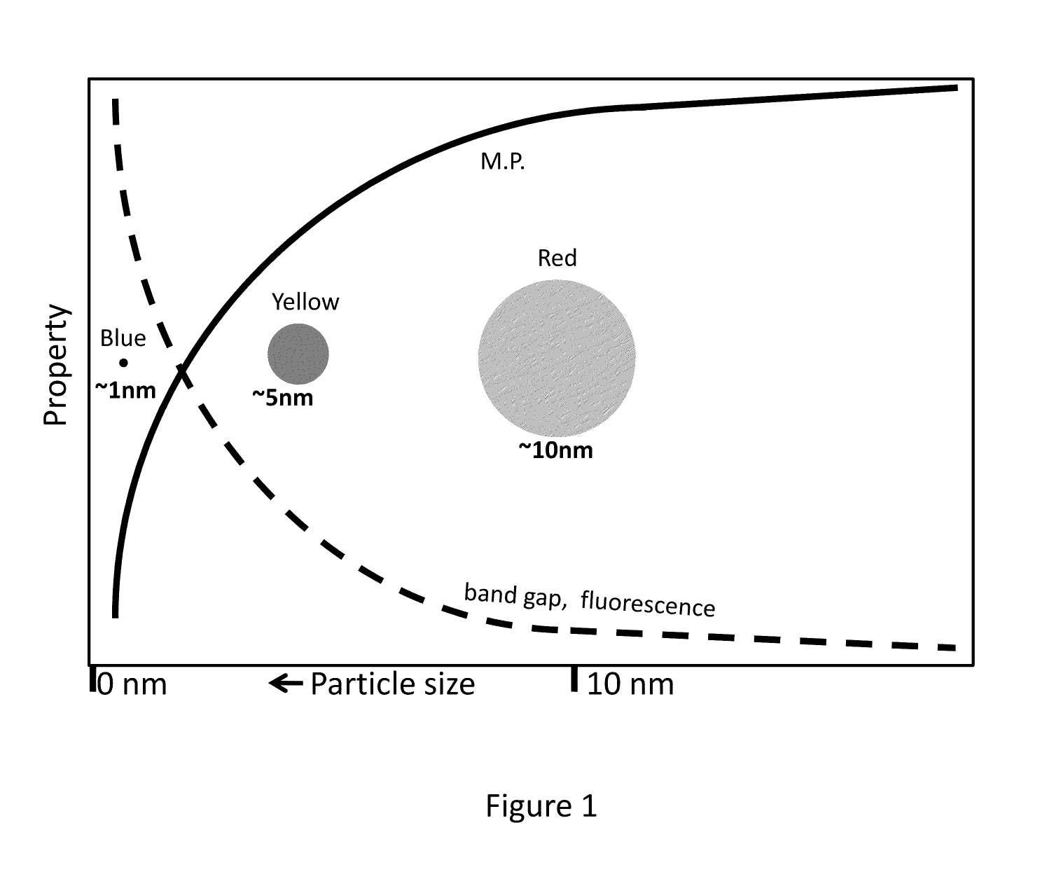

The invention can be described by reference to the Figures. A schematic presentation of changes in some properties of nano materials with the size of nanostructures is shown in FIG. 1. As the size of the nanostructures decreases there usually is a rapid and non-linear change in many properties such as the melting point, band gap, color, fluorescence, transparency and conductivity. A rapid change in these properties usually occurs below about 5 nm. Many metals such as gold, copper and silver undergo a rapid change in color and fluorescence as the size of the nano particle decreases.

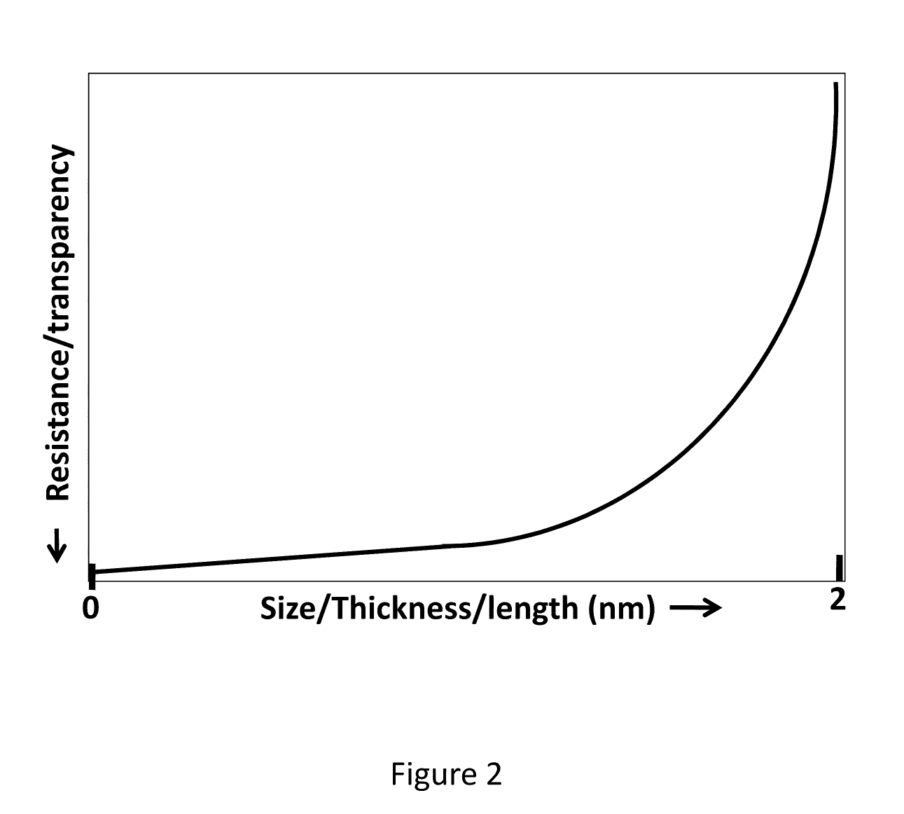

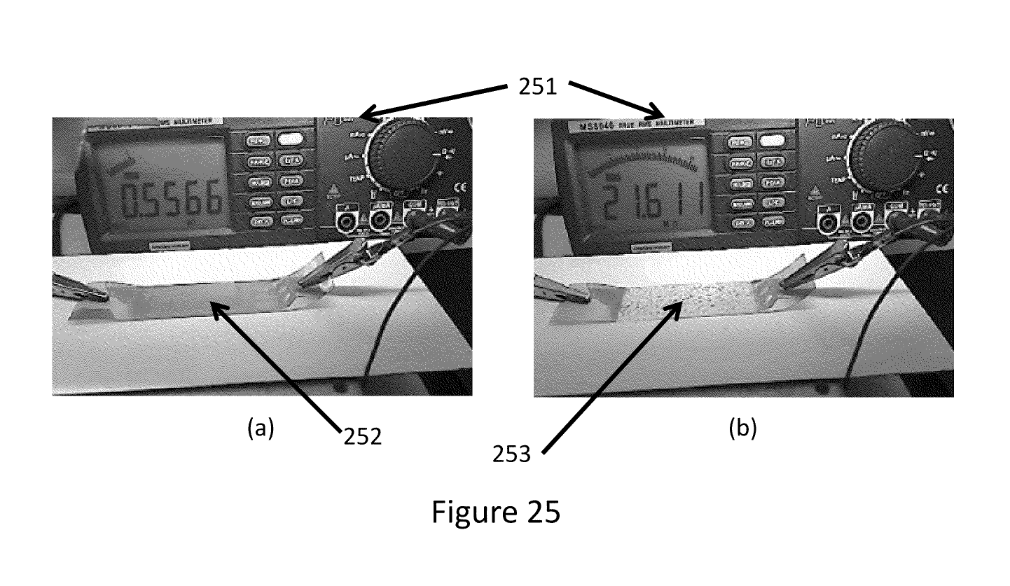

A schematic presentation of change in a property, such as transparency or electrical resistance with the thickness of a metallized (aluminized) plastic film or aluminum particles during an etching process is shown in FIG. 2. When the metal layer is destroyed, the product(s) formed is usually transparent with several orders of magnitude change in electrical resistance as shown in FIGS. 25a and 25b. As a nanostructure is destroyed, simultaneously there may or may not be the formation of another nanostructure (nanoproduct).

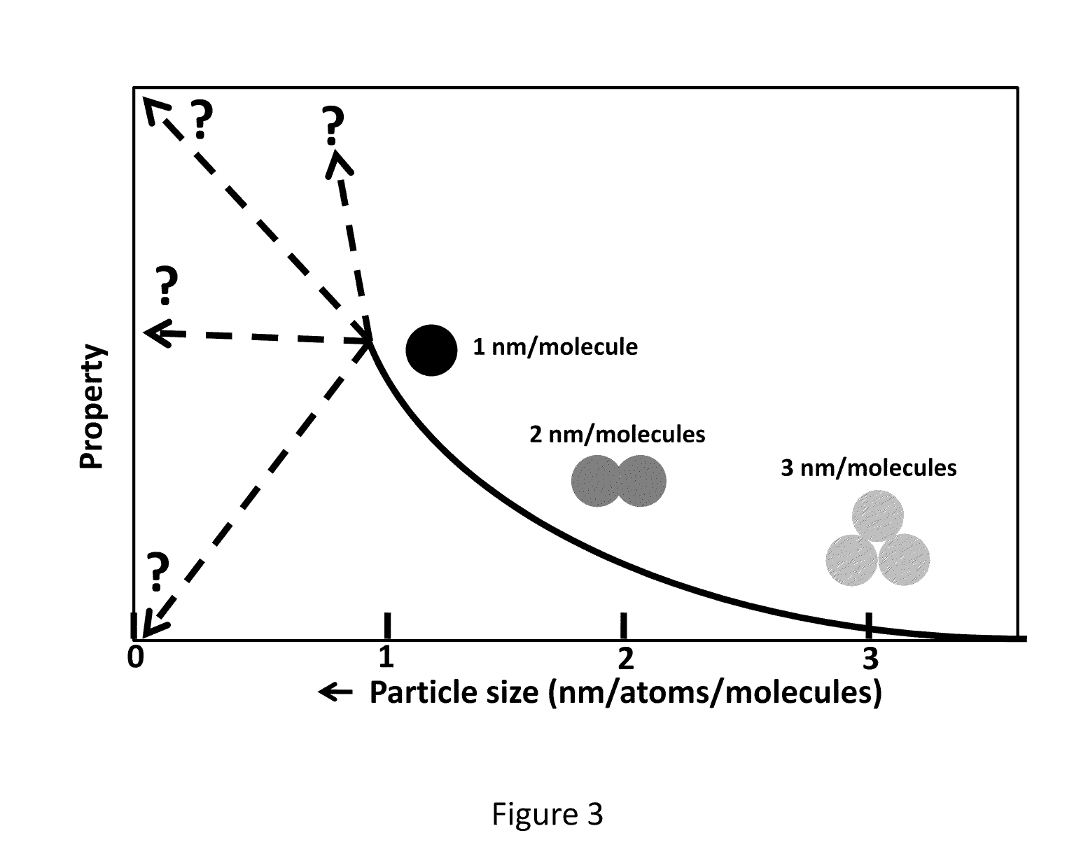

A schematic presentation of change in (disappearance or absence of) a property upon destruction of a nanostructure is shown in FIG. 3. When the smallest nanostructure is destroyed, the resultant product(s) can have a completely different set of properties (shown by arrow and question marks “?” in the Figure) from that of the nanostructure. As the particle size changes there is often a change in color and/or fluorescence with the change in the size of nanospheres. The nanostructure can have any shape, e.g., tube, fiber, rod etc.

In a reversed process, as small nanoparticles melt, fuse or coagulate/coalesce, there will be a change in properties, e.g., color/fluorescence.

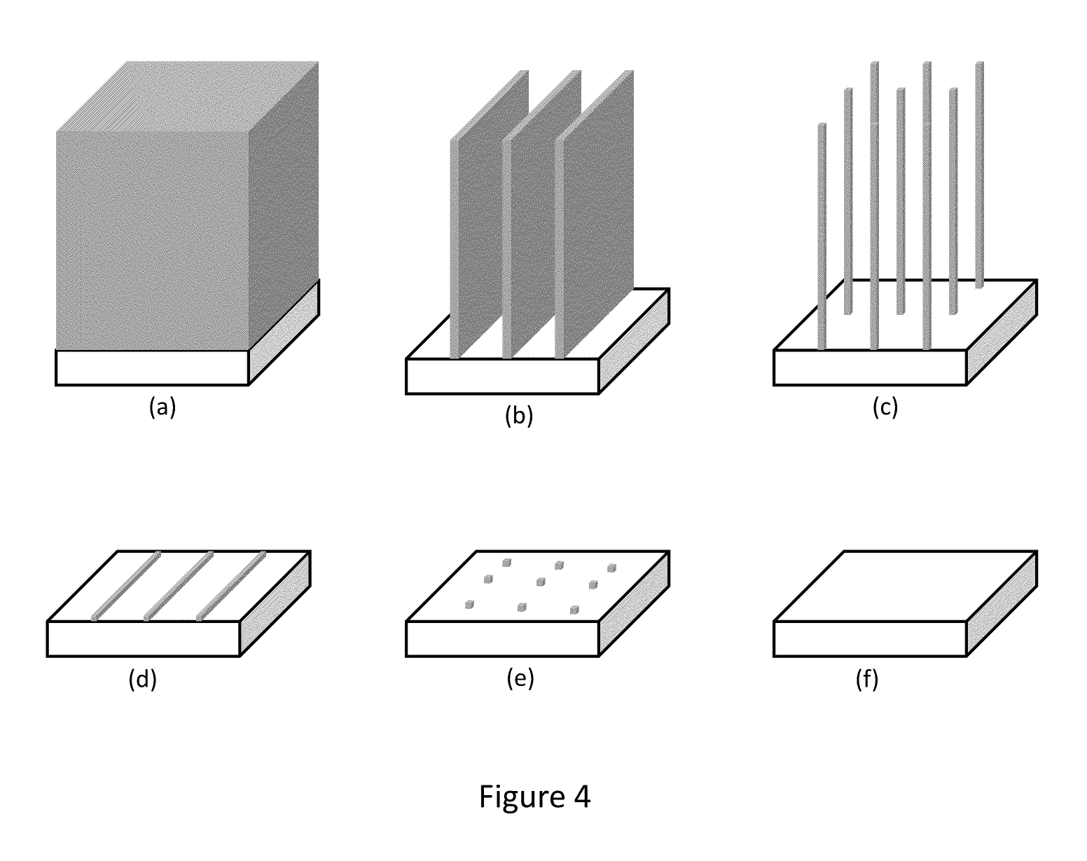

A schematic presentation of the creation of nanowalls (b), nanorods (c), thin nanofilms (d), nanowires/fibers (e) and quantumdots/nanodots (f) by selective etching of a nanofilm (a) on a substrate is shown in FIG. 4. These nanostructures can also have a coating of or be embedded in a protective/stabilizing material (including an activator, precursor or a transparent conductor). The activator can destroy these nanostructures. Some of these nanostructures can be created by selective etching. If the metal is an alloy, one can selectively etch one metal and create nanostructures of the other metal. These structures, for example, can be made by first coating the surface with a photo resist, imaging the resist and etching the metal. The nanostructures can be coated or embedded with many activators or their precursor.

The final nanostructure could be an atom or a molecule. Most likely it will be small number of atoms or molecules. If a nanostructure is reacted with a reactant, e.g., an etchant, it will reach a stage where the nanostructure will lose its nanodot or quantum dot properties. When such thermodynamically stable smallest nano (subnano) structure disappears, the properties of a nanodot completely disappear. If the product simultaneously forms another nanostructure, a new set of properties of the new nano will appear. Thus, disappearance of nanostructure will be associated an extreme change in one or more properties. This will be a unique case where there will be a rapid and dramatic change in the properties of a nanostructure as its size is reduced and then there will be a sudden disappearance of that property.

If a proper precursor is selected it will protect nanostructures from ambient conditions and react only to selected analyte.

Metallized plastic film of the desired thickness can be coated with an etch mask and etch the undesired portions. The substrate is usually a polymer/dielectric which could also be an un-etchable conductive material, such as gold.

Multiple nanostructures can be obtained by coating a etch mask with proper patterns followed by etching.

The nanostructure can coated with a dilute solution or by vacuum deposition of precursor to only cover the nanostructures. All nanostructures can have the same precursor coating.

The nanostructure can be completely covered with a precursor or coated with different precursors by a nanolithography technique. The nanostructures could be separate or joined. The nanowires can be completely covered with a precursor or coated with different precursors by a nanolithography technique.

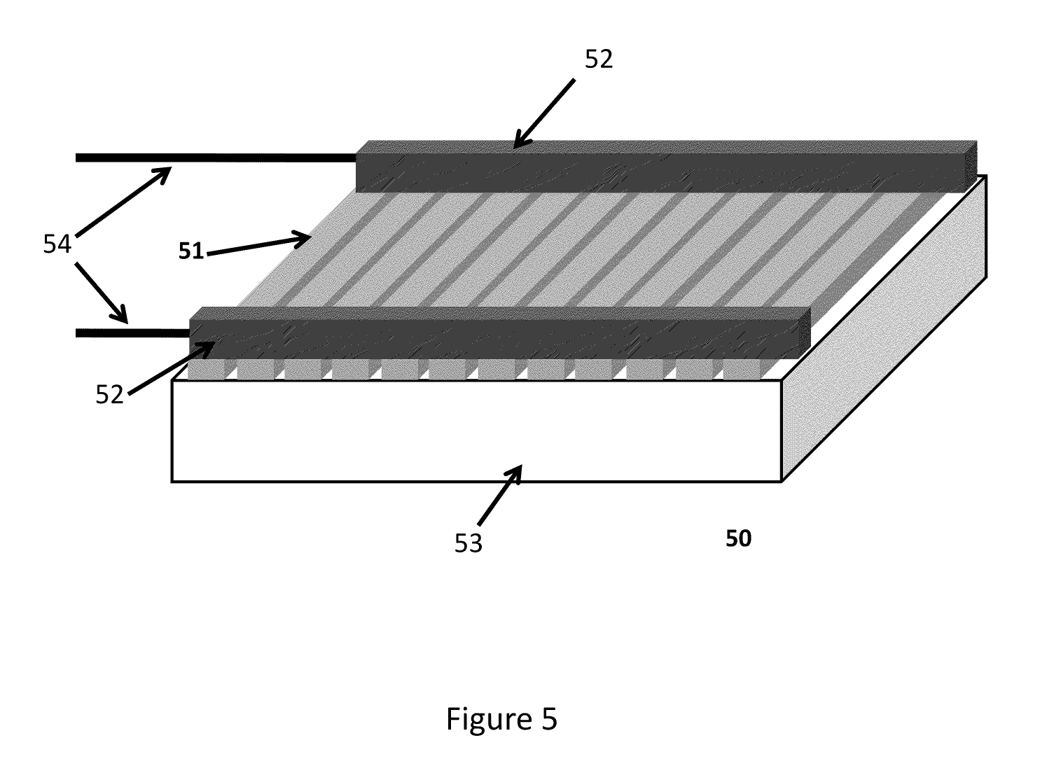

A schematic presentation of a dosimeter sensor device 50 made from nanowires 51 and two electrodes 52, with terminals 54, on a substrate 53 is shown in FIG. 5. The device may have coating of a precursor (not shown). The nanostructure can be any other than nanowires.

The terminals can be connected to an analytical instrument. The device can also be read with noncontact methods and instruments as well.

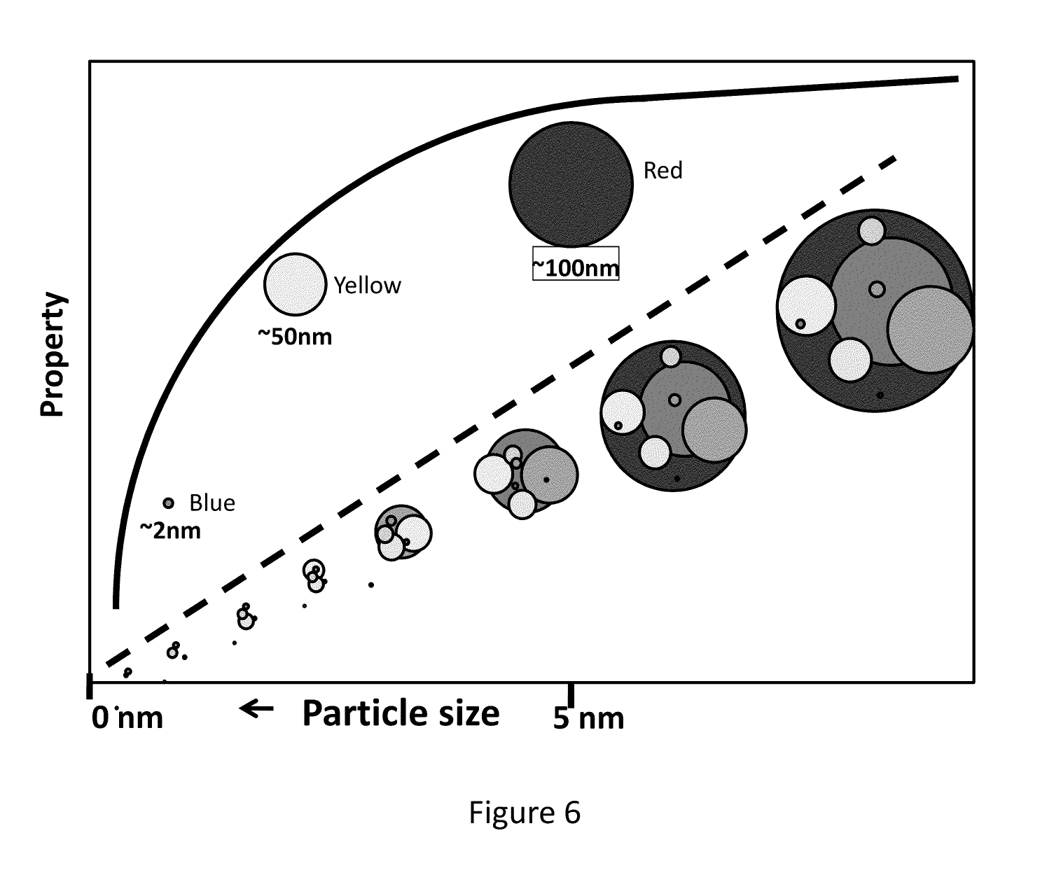

A schematic presentation of a change in property with the size of a nanostructure having a narrow size distribution (top curve) and with a broad random distribution (lower line) is shown in FIG. 6. By having a proper distribution of nanoparticles, a linear change in properties replaces an otherwise rapid change in properties. One can also use a broad and narrow distribution of the nanostructures. A variety of devices can be made by coating the broad or narrow distribution of nanostructures on a substrate. If required a binder, activator and precursor can be used.

When destructible nano structures have random distribution, they can provide a linear change in a property when etched/destroyed. A linear change in a property is desirable.

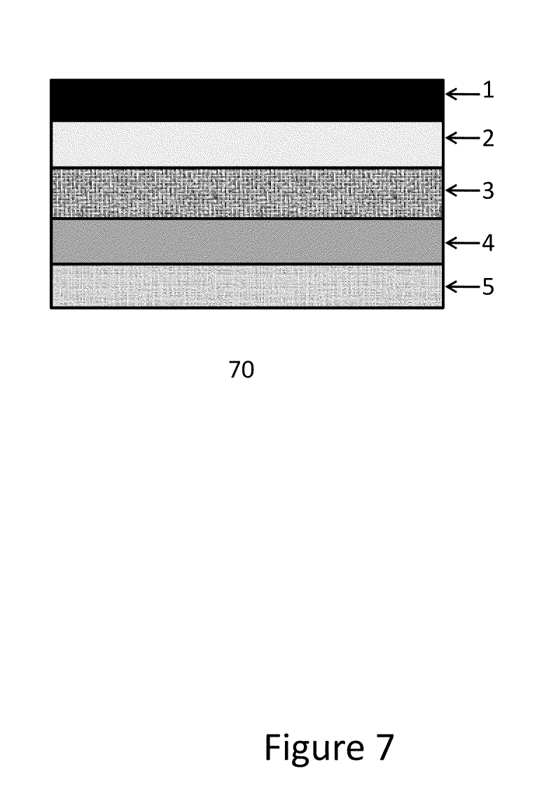

FIG. 7 shows a schematic cross sectional presentation of a dosimeter sensor 70 having a layer for transport/injection of electron 72, between cathode 71, and an electroactive layer 73, and a layer for transport of holes 74, between the electroactive layer and anode 75.

The device may have other layers, e.g., precursor or the electroactive layer may have a precursor.

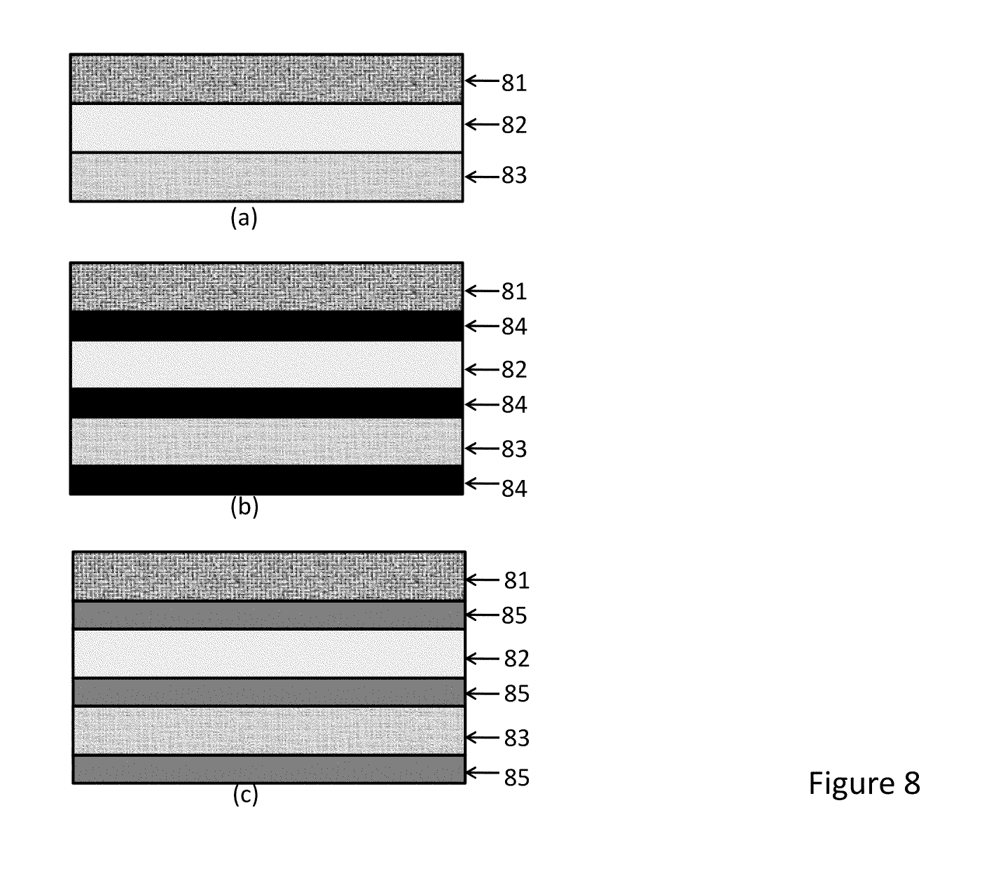

FIG. 8 shows a schematic cross sectional presentation of different layers of a dosimeter sensor devices made from different susceptible semiconductor layers 81, 82 and 83 having different semi-conducting properties, insulator/dielectric layers 85 and conductors 84.

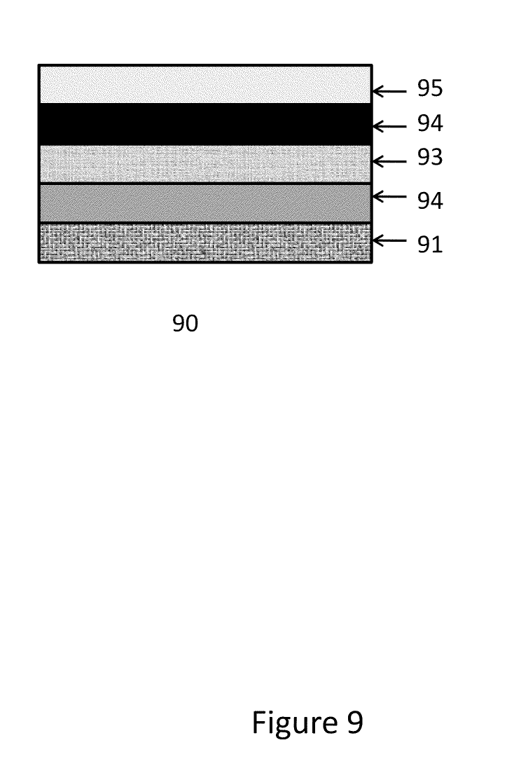

FIG. 9 shows a schematic cross sectional presentation of a de-activatable magnetic EAS system 90. The device can be composed of a substrate 91 having a layer susceptible hard nano magnet 92, a base 93, a susceptible soft nanomagnet 94 and a protective top layer 95. The properties of susceptible nano-magnets can be adjusted for the device. The magnets can have a coating of an activator or precursors (not shown).

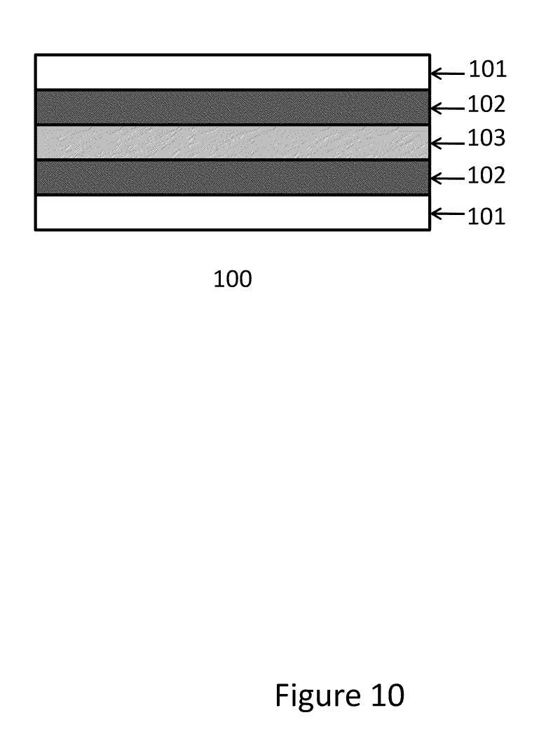

FIG. 10 shows a schematic cross sectional presentation of a pyro or piezo electric de-activatable transducer 100. The device can be made by a susceptible pyro or piezo electric nanostructure 103, sandwiched between two conductors which could be conductive indium tin oxide (ITO) 102 on a glass or plastic substrate 101.

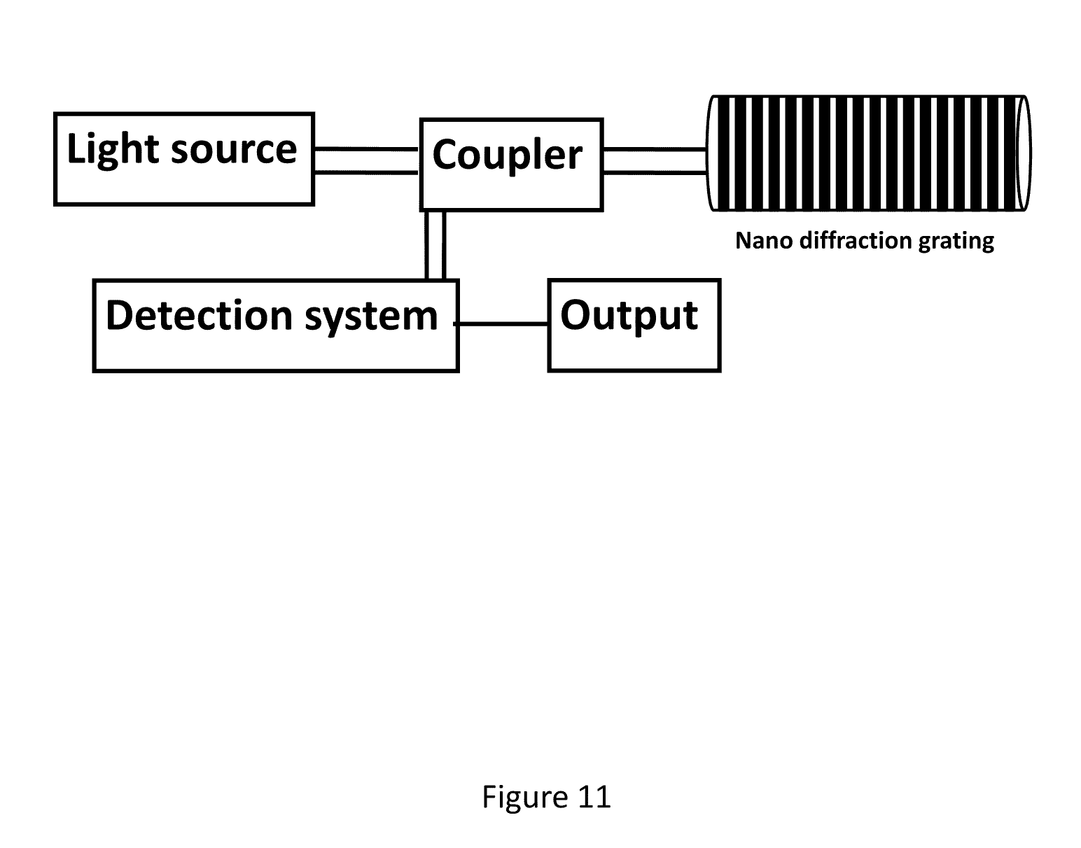

A flow chart of an apparatus having susceptible nano diffraction grating as a sensor is shown in FIG. 11. The grating sensor can be an optical fiber having a coating of a susceptible nanostructure. Any change in properties of grating can be monitored using a light source, coupler, photo detection system and a computer/monitor as an output system.

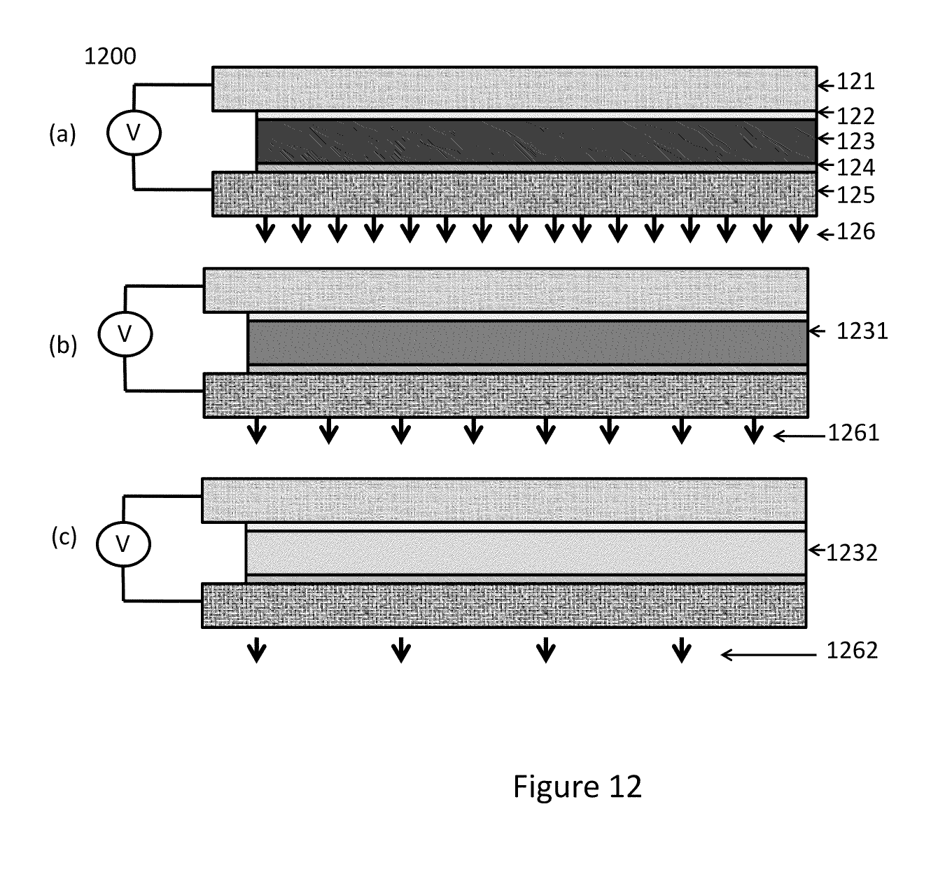

FIG. 12 shows a schematic cross sectional presentation of a dosimeter light emitting diode (LED) 120, having a susceptible phosphor layer 123, before a (a) medium (b) and high (b) dose of an analyte, such as X-ray. The phosphor 123 can have a dielectric layer 122 and an electrode 121 on one side and a transparent conductor 124 and a transparent substrate 125 on the other side. The LED will emit light 126 when connected to a proper power source. As the phosphor is susceptible to analyte/radiation such as X-ray, upon exposure to radiation, the phosphor will be damaged 1231, will be less effective in producing light and hence will emit less light, 1261. As the dose increases, the phosphor will become less effective, 1232 and will emit less light. The amount of light emitted can be measured by a photo-detector. Once calibrated for dose versus light emitted, one can determine the dose.

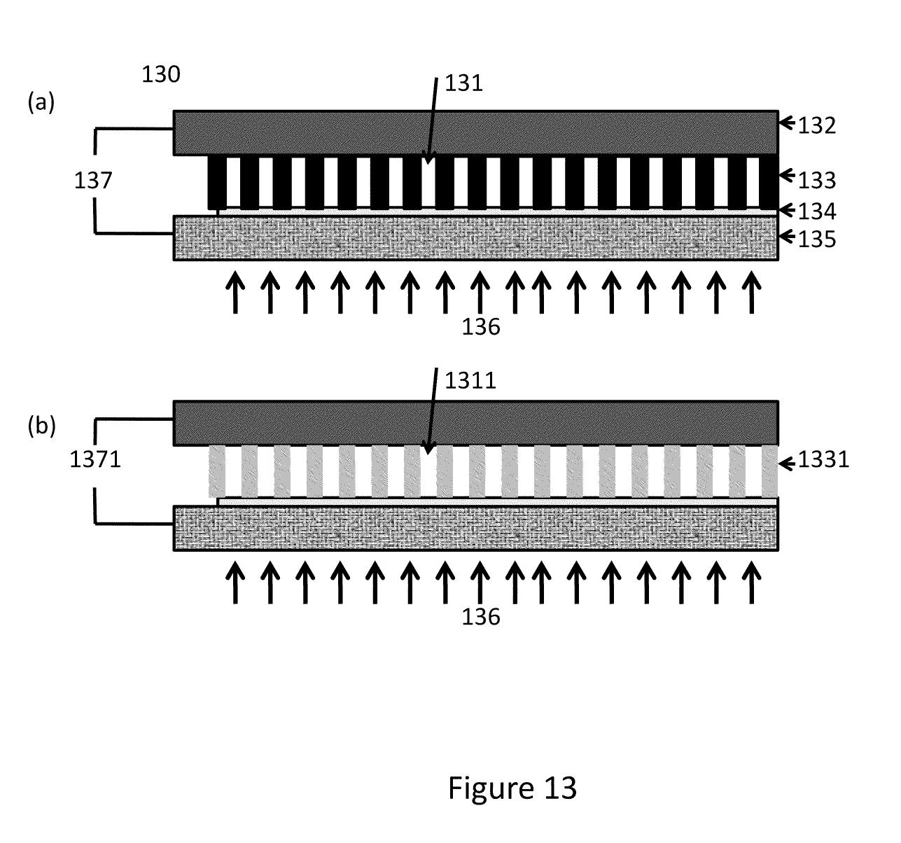

A schematic cross sectional presentation of a dosimeter photocell 130 having a susceptible photo absorbing layer before (a) and after (b) exposure to an analyte/radiation, such as X-ray is shown in FIG. 13. The dosimeter can be composed of a susceptible semiconductor 131 in a light absorbing layer 133 can have a transparent conductor 134 and a transparent substrate 135 on one side and an electrode for holes 132 on the other side. When exposed to a calibrated light source, the dosimeter photocell will generate current 137 which can be measured. Upon exposure to an analyte/radiation, the semiconductor nanostructures will be damaged 1311 and hence will produce less current 1371. Once calibrated for dose versus current produce, one can determine the dose.

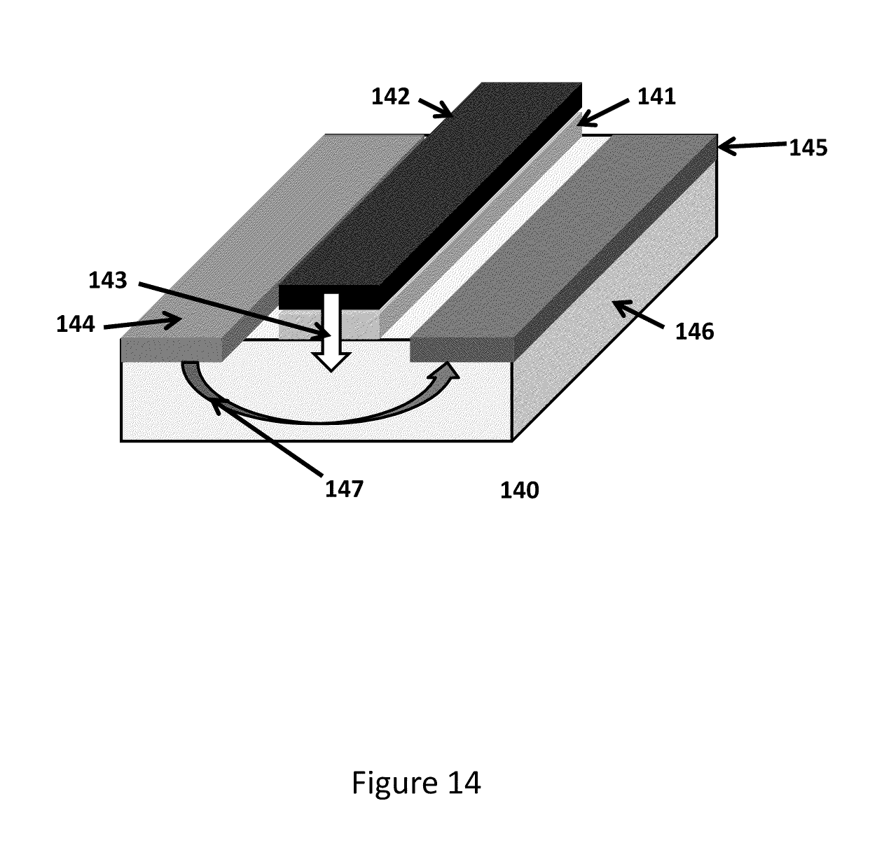

FIG. 14 shows a schematic presentation of a dosimeter/detector diode 140 having at least one susceptible component. The diode can be composed of an insulator 141, a gate 142, channel 143, source 144, drain 145 and a silicone wafer 146. The movement of electrons 147 will occur between the source 144 and the drain 145. If any destructible layer of the diode gets sufficiently damaged by an analyte such as radiation or a toxic agent, it will not function as a diode.

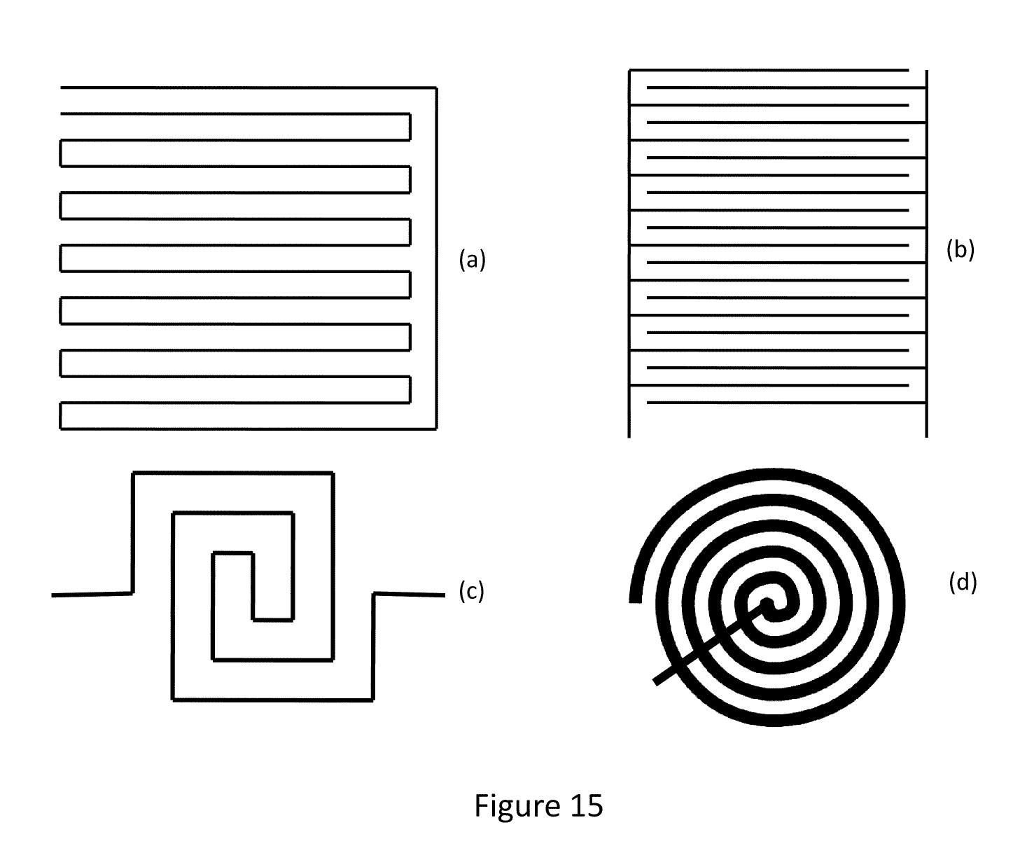

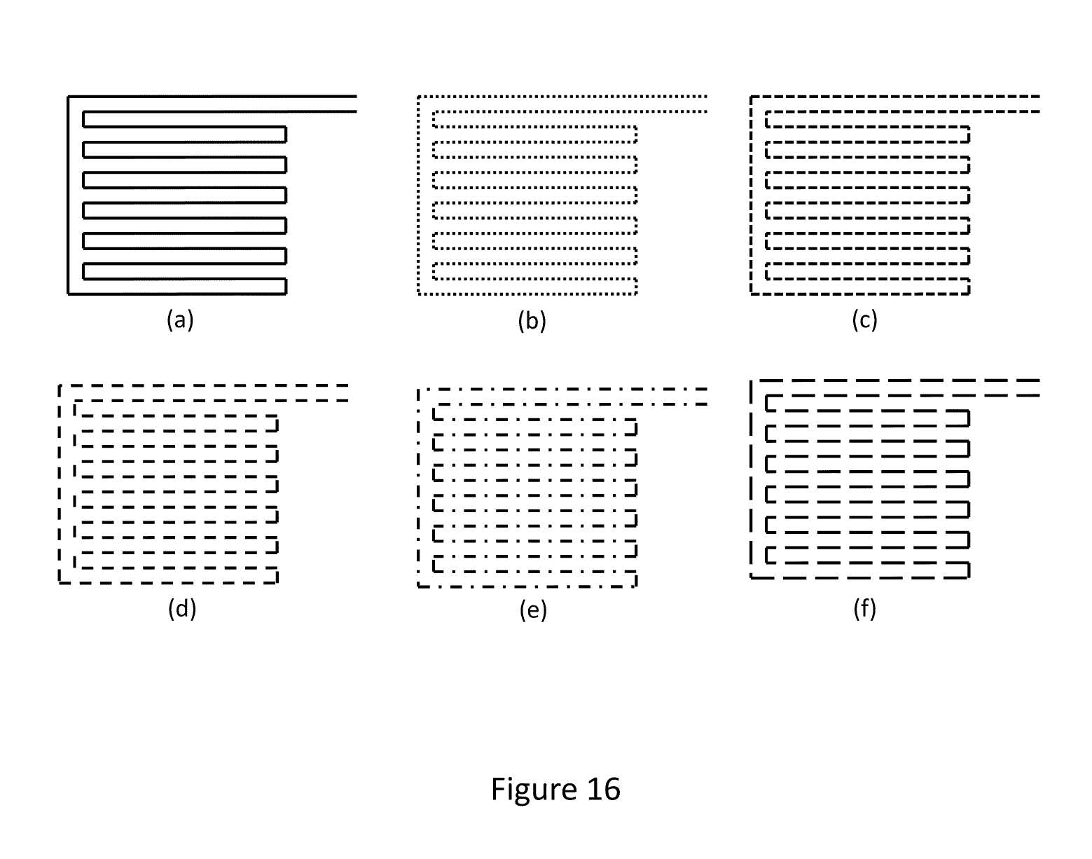

The antenna, electrodes or the sensors can have different shapes, sizes, configurations, arrangements and thicknesses as required. A schematic presentation of some representative examples of different types of susceptible nano antennas or sensors is shown in FIG. 15. The antenna, electrodes or sensors can be made from different nano materials, e.g., metals, semi-metals, semiconductors and non-metals depending upon the devices and processes. A schematic presentation of dosimeter nano antennas/sensors made from different susceptible materials for monitoring different agents is shown in FIG. 16.

A number of other shapes can also be used. The antenna, electrodes and sensors for example, can be in the form of a thin and flat square, triangle including those mentioned herein. The antenna can be made from a material destructible by an analyte.

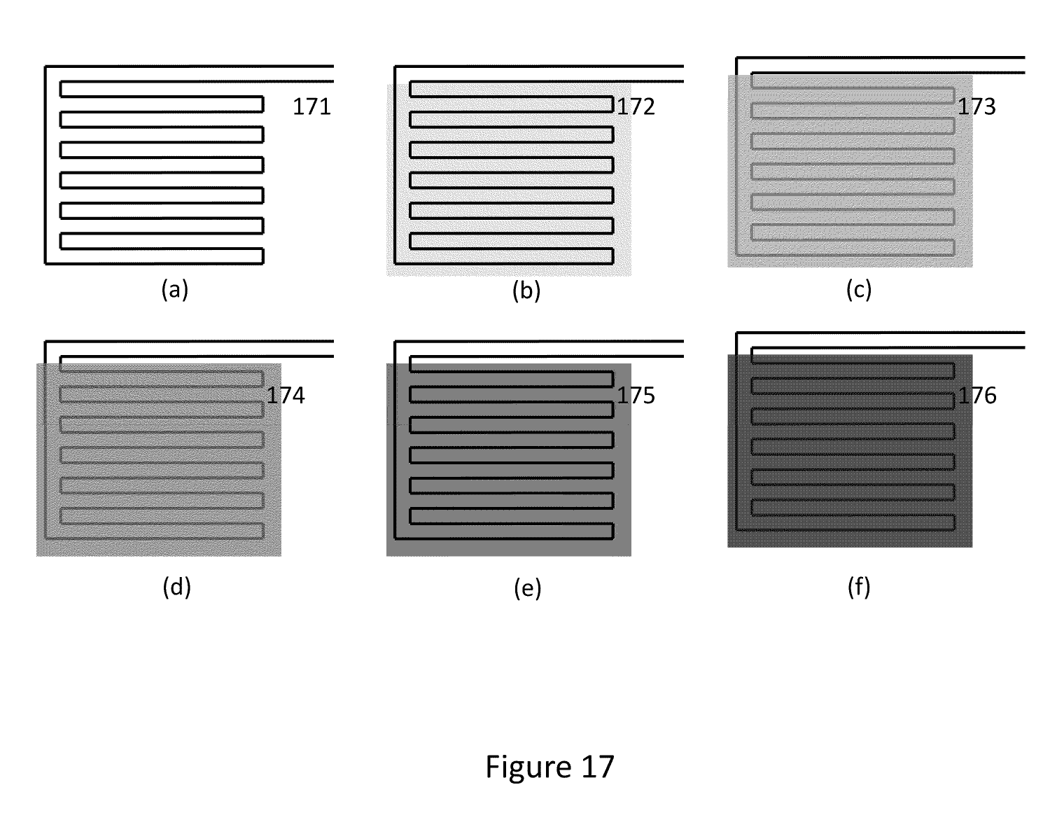

A schematic presentation of susceptible nano antennas/sensors coated with different precursors, 171-176, for monitoring different agents is shown in FIG. 17. The different precursors can be used for monitoring different analytes. For example, halocarbons can be used for monitoring radiation and humidity sensitive solid activators for monitoring humidity.

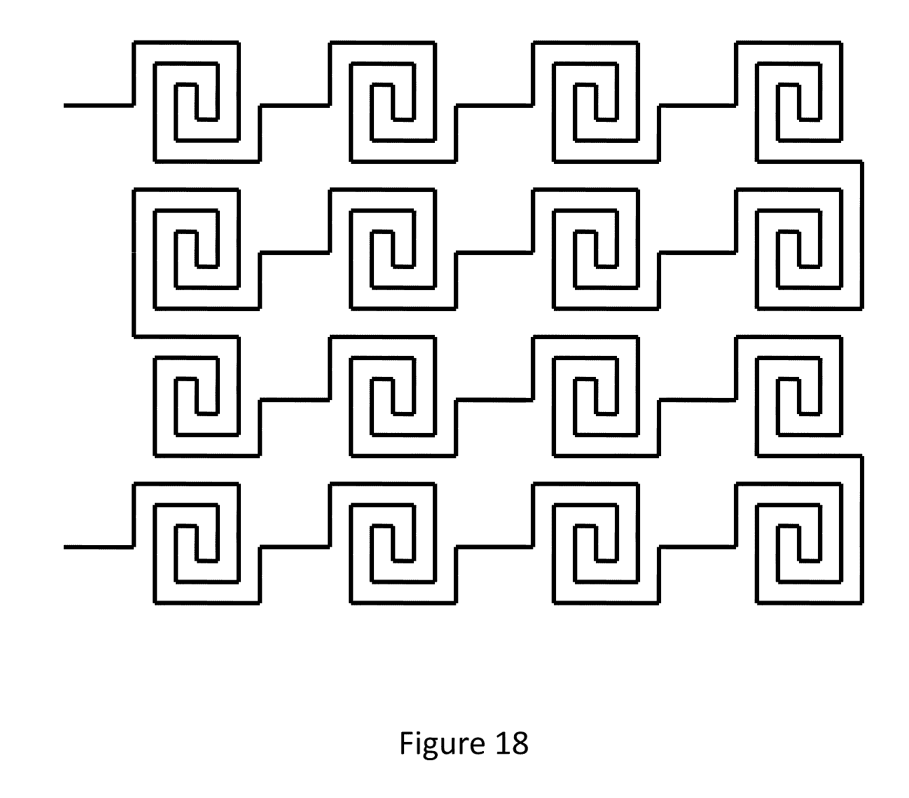

In order to increase the sensitivity of the devices, one can use more than one antenna/electrode in a series or parallel. A schematic presentation of a number of destructible nano antennas/ sensors connected in a series is shown in FIG. 18.

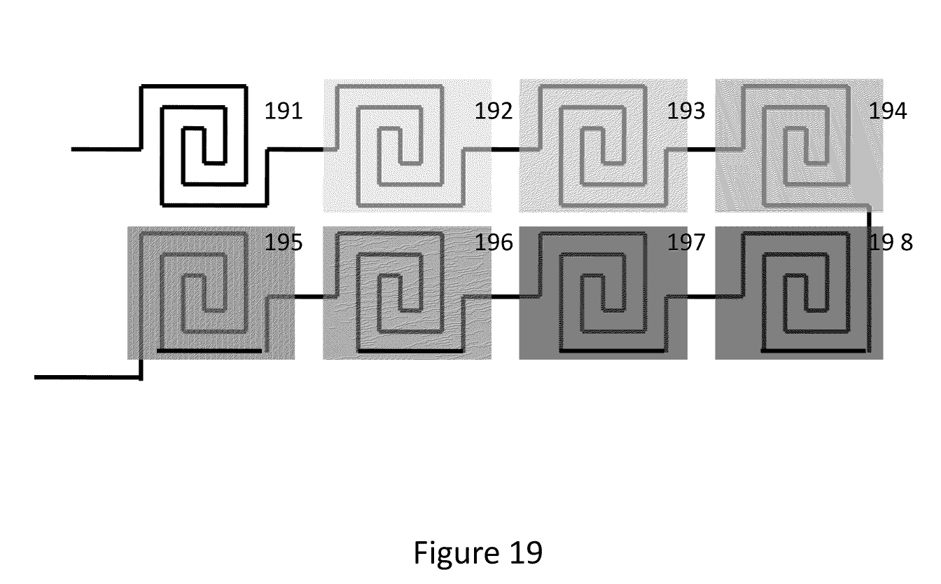

A schematic presentation of a number of destructible multisensory nano antennas/electrodes connected in a series and coated with different precursors 191-198 for monitoring different agents is shown in FIG. 19. Each antenna/sensor can be made individually addressable. The antenna can have different shapes. The antenna/electrodes can have electronic chips and circuitries as required. For example, RFID have an electronic chip and antenna.

Depending upon the nature of the antenna/electrodes one can monitor the change by contact or noncontact methods listed herein.

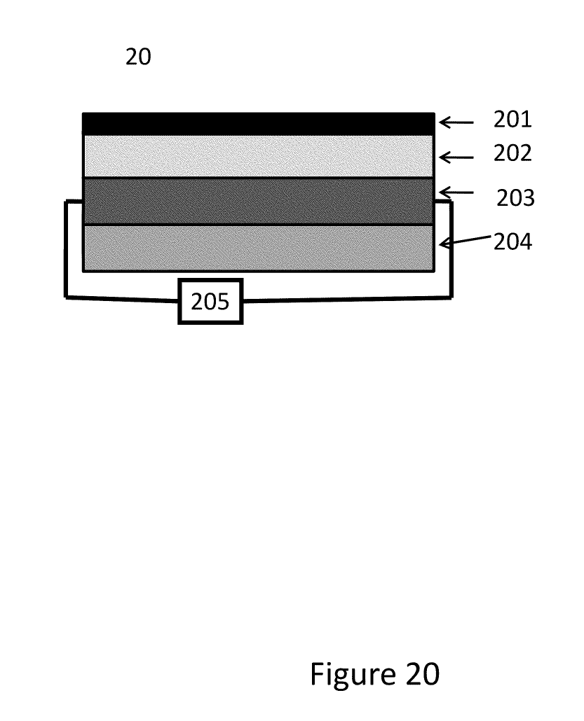

A schematic presentation of a dosimeter device, 20 for the measurement of change in parameters, such as resistance of a conductive or semiconductive nano layer, 203 on a substrate 204 upon exposure to analytes, such as high energy radiation, humidity and chemical agents is shown in FIG. 20. The device may have a protective or permeable layer 201. The analyte will interact/react with the precursor layer 202, and produce an activator. The activator will etch/destroy or reduce the measurable properties of the electrode or antenna 203. By measuring the change in properties of the electrode, one can measure the exposure to the analyte.

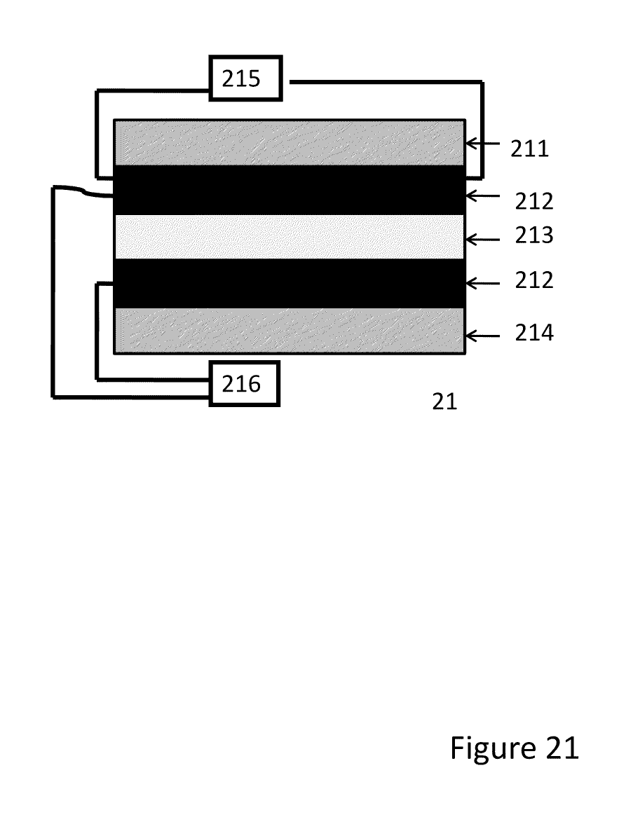

FIG. 21 shows a schematic presentation of a radiation dosimeter device (capacitor), 21 and apparatus for the measurement of change in more than one parameter, such as resistance 215 and capacitance 216 of a susceptible nano thin electrode 212 upon exposure to high energy radiation, such as X-ray. The device may have a protective layer 211 and a substrate 214. The precursor layer is sandwiched between the two electrodes. A demonstration of the concept is shown in Example 1. Upon reaction with analyte, the precursor will produce an activator which will react with the electrodes. The precursor layer is changing its dielectric properties, the capacitance will change and as the electrode is etched away and its resistance will change. Thus, by measuring the capacitance and resistance, one can measure the exposure to analytes more accurately.

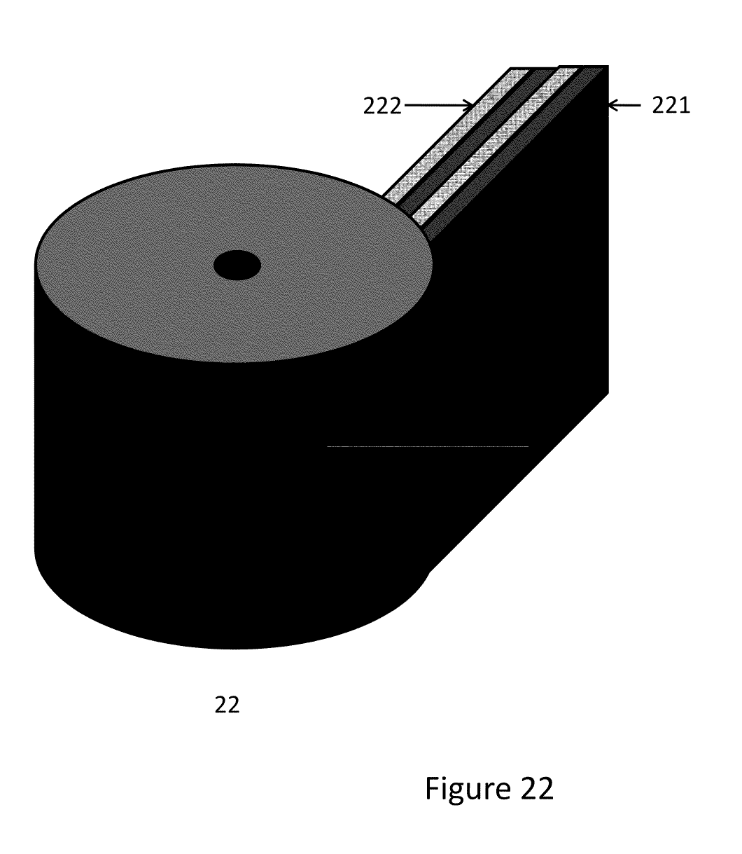

The capacitor type dosimeters can have a variety of known formats. One of them is a rolled capacitor. Because of the higher surface area, a roll capacitor will be more sensitive for monitoring lower concentration/exposure to analytes. A few of the designs are shown in FIGS. 22-24.

FIG. 22 show a schematic presentation of a radiation dosimeter/sensor in the form of a rolled capacitor 22 having two alternating layers of a susceptible nano thin electrode 221 and a dielectric layer containing a precursor 222.

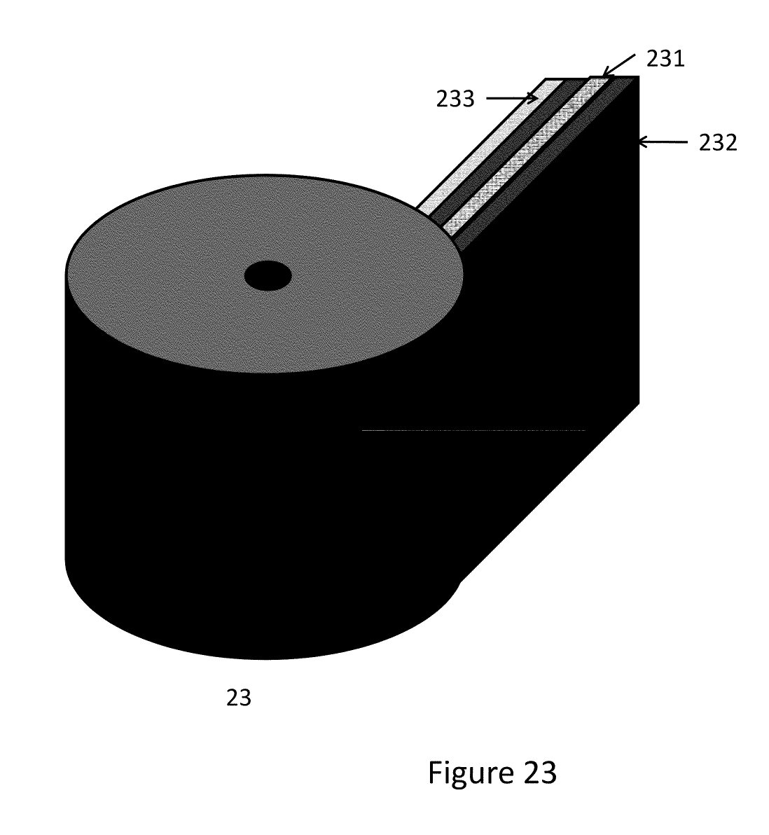

FIG. 23 shows a schematic presentation of a radiation dosimeter/sensor in the form of a rolled capacitor, 23 having a dielectric layer containing a precursor 231 between two layers of susceptible nano thin electrodes 232 and a stable dielectric layer 233.

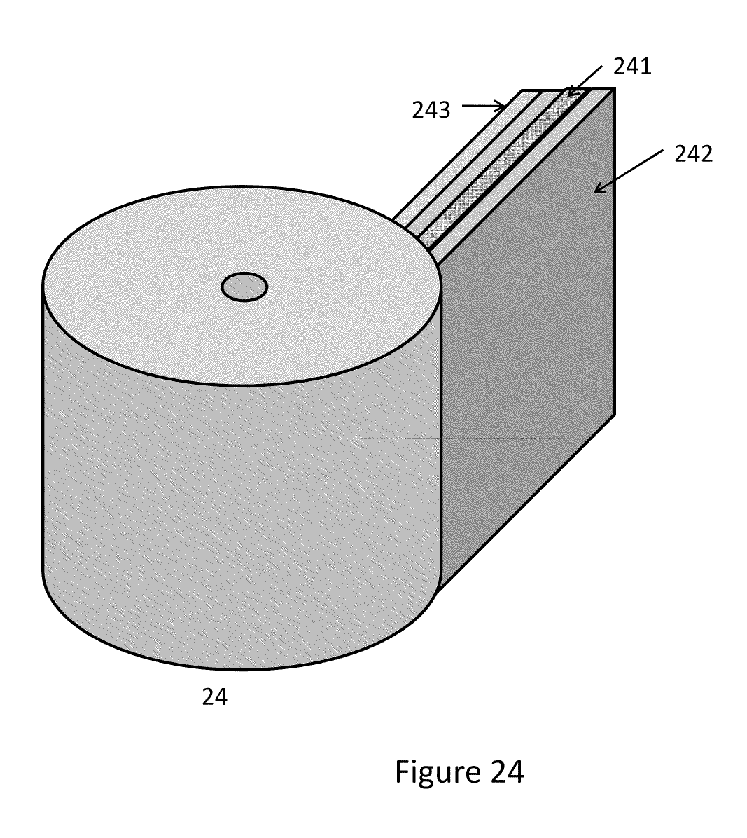

FIG. 24 shows a schematic presentation of a radiation dosimeter/sensor in a form of a rolled capacitor, 24 having a dielectric layer containing a precursor 241 between two layers of non-destroyable thin electrodes 242 and a stable dielectric layer 243.

FIG. 25a is a photograph of an experimental setup for the determination of change in electrical resistance of a metallized PET (polyester) film as a susceptible/destroyable electrode having a thin coating of a precursor (a halocarbon) and then exposed to short wavelength UV light. FIG. 25b is a photograph of the device of FIG. 25a after 2.5 hrs of the UV exposure. Electrical resistance changed from 0.56 kilo Ohms (FIGS. 25a) to 21.6 mega Ohms. The electrode and matching container can be any shaped flat, square, folded, zigzag, cylindrical, spiral, etc. The precursor, e.g., halo-compound can be liquid, emulsion, viscous liquid, gel, dry coating, paste, etc. Typically, the conductor can be a metallized plastic film. The container is preferred to be opaque but can be transparent with a UV absorber, i.e., as long as not affected by light. The change in resistance upon radiation can be measured by direct contact or non-contact techniques. The preferred destructible metals are aluminum, zinc and copper. Once the oxide layer is destroyed by an acid or base, water can destroy some of the metals such as aluminum.

Though the change in properties is explained using a specific nanostructure, such as rod, dot, sphere, film in the figures above, the nano structure could be any other proper structure suitable for the application.

### Analytical Methods:

In order to determine a change in a property of a nanostructure and a device there from, one can use one or more analytical methods. One or more of the following analytical methods can be used for determining change in destructible and non-destructible nanostructures: Cyclic voltammetry, electron paramagnetic resonance (EPR) also called electron spin resonance (ESR), energy dispersive spectroscopy, ion selective electrode, e.g., determination of pH, refractive index, resonance enhanced multiphoton ionization, magnetic susceptibility, atomic fluorescence spectroscopy, attenuated total reflectance, cathodoluminescence, dielectric spectroscopy, dynamic vapor sorption, differential reflectance spectroscopy, electroluminescence, electrophoretic light scattering, electron nuclear double resonance, electron paramagnetic resonance spectroscopy, fluorescence correlation spectroscopy, fluorescence cross-correlation spectroscopy, glow discharge mass spectrometry, glow discharge optical spectroscopy, ion neutralization spectroscopy, low-energy ion scattering, nuclear magnetic resonance spectroscopy, optical beam induced current, optically detected magnetic resonance, optical emission spectroscopy, photocurrent spectroscopy, potentiodynamic electrochemical impedance spectroscopy, porosimetry, resonant inelastic X-ray scattering, resonance Raman spectroscopy, thermoacoustic tomography, total internal reflection fluorescence microscopy, total reflection X-ray fluorescence analysis, ultrasound attenuation spectroscopy, ultrasonic testing, X-ray diffuse scattering, X-ray photoelectron emission microscopy, X-ray photoelectron spectroscopy, X-ray reflectivity, X-ray diffraction, X-ray Raman scattering, X-ray fluorescence analysis, X-ray standing wave and hybrid or modified techniques of these methods. The method(s) used depend upon many parameters, such as nanostructure, reaction of nanostructure and agent.

Most of the above methods also have several other divisional methods. For example, electroanalytical methods includes adsorptive stripping voltammetry, amperometric titration, anodic stripping voltammetry, bulk electrolysis, cathodic stripping voltammetry, chronoamperometry, coulometry, cyclic voltammetry, differential pulse voltammetry, Electrogravimetry, linear sweep voltammetry, normal pulse voltammetry, Polarography, potentiometry, rotated electrode voltammetry and staircase voltammetry.

Similarly, most of the above methods and instruments have parts. For example, electroanalytical analysis instruments can have auxiliary electrode, dropping mercury electrode, electrolytic cell, galvanic cell, hanging mercury drop electrode, ion selective electrode, mercury coulometer, potentiostat, reference electrode, rotating disk electrode, rotating ring-disk electrode, salt bridge, saturated calomel electrode, silver chloride electrode, standard hydrogen electrode, ultramicroelectrode and working electrode.

Similarly there are many theories for each method listed above. It is the beyond the scope of this application to even list all analytical instruments, methods, their parts and theories that can be used for the inventions disclosed herein.

Though destructive and direct contact methods can be used, preferred methods and instruments are those which determine change in properties without destroying the sensor and non-contact.

It is an object of the invention to use or modify these methods or their hybrids, create their hybrid for monitoring an agent using a destructible and non-destructible nanostructure. For example, once an agent reacts with a thin conductive or metal layer or precursor for activator, it can produce compounds which can be monitored with one or more of these methods. The metal or oxide on it can act as a catalyst to produce chemicals which can be monitored by one or more of these methods. These methods are described in detail in a number of books and reviews. The above and other analytical techniques and instruments can be used for monitoring change in properties of nanostructures for the applications/dosimeters disclosed herein.

### Electroanalytical Methods:

Electroanalytical methods which measure the potential (volts) and/or current (amps) in an electrochemical cell containing an analyte can be used for the present inventions. These methods that can be used can be categorized according to which aspects of the cell are controlled and which are measured. The three main categories are potentiometry (the difference in electrode potentials is measured), coulometry (the cell's current is measured over time), and voltammetry (the cell's current is measured while actively altering the cell's potential). It is an object of this invention to use these methods, their modifications, variations and also their hybrids using a nanostructure, especially destroyable nanostructure e.g., a thin layer of a reactive metal and a protective or detector/precursor layer on the metal which undergo at least one change in measurable property.
Electrode: A substrate having a nanolayer of a conductive nanomaterial can be used as an electrode or electrochemical sensor for one or more of electroanalytical and non-electroanalytical techniques described herein. The electrode can be substantially destructible. The conductive layer is also referred herein as a metal, organic metal and/or semiconductive layer. The conductive nano film can be converted to other nanostructures by selective etching and other methods. The substrate for the electrode could be opaque, translucent or transparent. The electrode, the metal layer and the substrate could be of any shape, e.g., a very thin film/coating, fiber, rod, flat, patterned, hollow, folded, spiral, zigzag, wounded or rolled, cylindrical, any irregular shape and addressable. They can be zero (e.g. nanodots), one (e.g., thin fiber), two (e.g., thin film) or three dimensional. The substrate could be an insulator, semi-conductor, semi-metal, metal or their alloy. The preferred substrate is plastic or glass. The substrate could be porous. The electrode could be mono, bi or multi-layered. The thickness of the metal or the conductive layer can be from a few Angstroms to a micron, preferably 10-1,000 Angstroms. A metallized plastic film can be used as an electrode. The metal layer can be porous, continuous or particulate. The electrode could be in form of a hologram or grating.

Carbon, activated, charcoal, film, fiber, etc can be used as an electrode. Transparent conductors, such as indium tin oxide can also be used as an electrode. The electrode can be porous or micro-textured.

Aluminum, copper and their alloys can be coated on highly resistive metal or alloy for an electrode. This allows one to measure properties even when the nanostructure is destroyed. Electrochemical means of quantifying or detecting an analyte is one of the preferred methods because of their simplicity, both in terms of device manufacture and in terms of the ease of use. Electrochemical sensors have often been in the form of either potentiometric or amperometric devices. Potentiometric devices measure the effects of the charges on atoms and their positions; examples include the chemFET (chemical field effect transistor) and the ion-selective electrode (including pH electrodes). Amperometric devices operate on the principle of applying a potential and measuring the resulting current, where the magnitude of the current generated is usually related to the amount of analyte present; alternatively, the total charge passed over a time may be used to represent the amount of analyte in a region of the sample. Because the range of compounds that can generate electrochemical currents is smaller than those that carry charges, amperometric devices can often offer greater selectivity.

The presence of an analyte in the sample is evaluated in an electrochemical system using a conduction cell-type apparatus. A potential or current will be generated between the two electrodes of the cell sufficient to bring about oxidation or reduction of the analyte or of a mediator in an analyte-detection redox system, thereby forming a chemical potential gradient of the analyte or mediator between the two electrodes. After the gradient is established, the applied potential or current is discontinued and an analyte-independent signal is obtained from the relaxation of the chemical potential gradient. The analyte-independent signal can be used to correct the analyte-dependent signal obtained during application of the potential or current. This correction allows an improved measurement of analyte concentration because it corrects for device-specific and test specific factors, such as transport (mobility) of analyte and/or mediator, effective electrode area, and electrode spacing (and as a result, sample volume), without need for separate calibration values.

The cell or electrochemical cell may have a reference electrode.

A substrate having a nano-structure, e.g., a thin layer of a conductive material, such as metal, organic metal or semiconductor having one or more of (1) a naturally or artificially applied protective, permeable or absorbent/adsorbent layer, (2) layer of an activator, its precursor, catalyst or modulator can also be used as electrode or electrode assembly. The protective layer can be a naturally formed or intentionally added oxide layer or any other layer, such as phosphate, zincate, chromate, etc. Electrochemical electrode/detectors can be used in mobile detectors to detect blister, nerve, blood, and choking agents.
Thermoelectric conductivity. The electrical conductivity of certain materials can be strongly modulated following the surface adsorption of various chemicals. Heated metal oxide semiconductors and room-temperature conductive polymers are two such materials that have been used commercially. The change in sensor conductivity, especially when the electrode is a destroyable nanostructure or undergoes a change in conductivity, can be measured using a simple electronic circuit, and the quantification of this resistance change forms the basis of sensor technology.
Destruction of electrode: When exposed to an agent in a gas or liquid state, the agent will first react with the nano thin oxide/protective layer, if any, and then with the metal or conducting nanolayer. Thus, this type of reactive electrode will decay as the reaction proceeds. These type of electrodes or nanolayers which degrade, decay, perish, corrode, rot, putrefy, decompose, crumble, disintegrate, deteriorate, destruct, become unstable or de-metallize, undergo some change in physical or chemical properties are referred herein as destroyable electrode/sensor/nanostructure.
Destruction of oxide layer: If a metal has an oxide layer, it can be removed, thinned, changed, and made permeable to an agent by adding an agent which selectively reacts with the oxide layer. The preferred reagents are chelates. The oxide layer can be opaque, transparent, permeable, semi-permeable, selectively permeable, reactive or destroyable.

For indicating devices disclosed in out U.S. patent application Ser. No. 12/478,232, the oxide layer can be obtained by vacuum evaporation of metal under controlled atmosphere of oxygen, where metal gets oxidized and an oxide layer is deposited on the said indicating layer. By selecting a proper metal or an alloy, one can minimize or eliminate the formation of the oxide layer or impermeable oxide layer. For certain transparent conductive layers, such as that of indium-tin-oxide and antimony tin oxide, the conductive and the oxide layers will be the same. The metal nanolayer of the electrode may have an oxide layer. The metal and oxide layer can be on, one, both or all sides the substrate. The metal layer may have one or more additional organic, inorganic or organo-metallic layers, e.g., protective or selective, e.g., semi-permeable layer. The extra layer can be an absorbent, adsorbent, super absorbent or super adsorbent material, especially polymeric material.

The nanolayer of the electrode may have a layer of an activator, pre-cursor, catalyst, promotor, additive, retarder, reactant or co-reactant. Some of the activators, precursors, catalysts, promotors, reactants and co-reactants are listed, define or described in our U.S. patent application Ser. No. 12/478,232 and cited herein as reference. Water or other solvents/liquids or ionic liquids can be used as a media, catalysts, facilitator or modulator. The media could be solid, liquid, semi-solid, gel, emulsion, gas or plasma.

As used herein, the term “conduction cell” or “conductivity cell” refers to a device comprising two electrodes in contact with a medium (e.g., air, gas, solution, gel, solid), such that the conductance of the medium can be calculated by passing current between the electrodes.

As used herein, the term “effective electrode ^{area} refers to the electrode area that is in electrolytic/activator/precursor contact with the sample. The effective electrode area may be varied by altering the geometry of the electrode or by partial contact of the electrode to the sample.

As used herein, the term “electrolytic contact” refers to having an electrochemical system comprised of at least one electrode deployed in a manner so as to gather electrochemical information from a sample. Examples include, but are not limited to, an electrode in physical contact with a sample; an electrode separated from a sample by a membrane, a film, or other material; and an electrode separated from a sample by an aqueous medium. Examples of electrochemical information include Faradaic current, nonfaradaic current and chemical potential.
Surface treatment and pre-treatment of electrode: If required, the electrode surface can be pretreated to destroy the naturally oxide or similar protective layers, effect or phenomenon. For example, expose the electrode to initial dose of radiation till the oxide layer is destroyed by a precursor. One can protect the surface of the electrode with a layer which is gets readily destroyed when the electrode is dipped or exposed to a media, environment or agent. This can also be done by selecting a metal or an alloy or amalgam which either does not form an oxide layer or forms a very thin layer, monolayer which is permeable to precursor. The surface can be protected by a very vulnerable layer, such as monolayer which gets destroyed when the system is activated. Alternatively, one can pre-treat the surface with an agent for example, chlorine or similar agents so that the protective oxide layer is easily destroyed and/or converted to permeable layer.
Devices having electrode: The devices which require at least one electrode, especially high electrical conductivity and optical transparency include, but are not limited to, touch screens (e.g., analog, resistive, 4-wire resistive, 5-wire resistive, surface capacitive, projected capacitive, multi-touch, etc.), displays (e.g., flexible, rigid, electro-phoretic, electro-luminescent, electrochromatic, liquid crystal (LCD), plasma (PDP), organic light emitting diode (OLED), etc.), solar cells (e.g., silicon (amorphous, protocrystalline, nanocrystalline), cadmium telluride (CdTe), copper indium gallium selenide (CIGS), copper indium selenide (CIS), gallium arsenide (GaAs), light absorbing dyes, quantum dots, organic semiconductors (e.g., polymers, small-molecule compounds), solid state lighting, fiber-optic communications (e.g., electro-optic and opto-electric modulators) and microfluidics (e.g., electrowetting on dielectric (EWOD). These devices will not function if the electrode is destroyable or get destroyed by an agent, such as chemical or radiation. Hence, one can monitor an agent by determining non-functionality, limited functionality or abnormal functionality of these devices. Many other analytical techniques and equipment can be used including those listed herein.
Ion Mobility Spectrometry (IMS): IMS operates by drawing air at atmospheric pressure into a reaction region where the constituents of the sample are ionized. The ionization is generally a collisional charge exchange or ion-molecule reaction, resulting in formation of low-energy, stable, charged molecules (ions). The agent ions travel through a charged tube where they collide with a detector plate and a charge (current) is registered. A plot of the current generated over time provides a characteristic ion mobility spectrum with a series of peaks. The intensity (height) of the peaks in the spectrum, which corresponds to the amount of charge, gives an indication of the relative concentration of the agent present. This technology is mainly used in mobile detectors to detect nerve, blister, and blood agents. If the charged tube and detector plates are thin conductive and are or have reactive/destroyable nanolayers, they will react with an agent and get destroyed as the reaction proceeds and the spectrum and many other properties will change irreversibly. It is an object of this invention to modify the IMS technique by replacing the charged tube and detector plates with thin conductive and reactive/destroyable nanolayers.
Photo Ionization Detectors (PIDs): PIDs operate by passing the air sample between two charged metal electrodes in a vacuum that are irradiated with ultraviolet radiation, thus producing ions and electrons. The negatively charged electrode collects the positive ions, thus generating a current that is measured using an electrometer-type electronic circuit. The measured current can then be related to the concentration of the molecular species present. If the charged electrodes are thin conductive and reactive/destroyable nanolayers, they will react with an agent and get destroyed as the reaction proceeds.
Color-Change indicators: This technology is based upon chemical reactions that occur when an agent interacts with various chemicals (either in solution) or coated on a substrate. The most common indicator (for a positive response) is a color change. Detection tubes, papers, or tickets use some form of surface or substrate to which a reagent solution is applied.

At nanolevel, these indicating materials will be much more sensitive and a color change can occur from UV to IR. Many of these indicators will undergo a change in fluorescence along with the color change. Color change can be monitored visually as well as with a spectrophotometer.

It is an object of the present invention to prepare nanolayers of chemicals which react with chemical and biological agent and undergo an irreversible change in color or fluorescence or by other methods listed herein.
Ellipsometry: The name “ellipsometry” stems from the fact that the most general state of polarization is elliptic. Upon the analysis of the change of polarization of light, which is reflected off a sample, ellipsometry can yield information about layers that are thinner than the wavelength of the probing light itself, even down to a single atomic layer. Ellipsometry can probe the complex refractive index or dielectric function tensor, which gives access to fundamental physical parameters and is related to a variety of sample properties, including morphology, crystal quality, chemical composition or electrical resistance. It is commonly used to characterize film thickness for single layers or complex multilayer stacks ranging from a few angstroms or tenths of a nanometer to several micrometers with an excellent accuracy.

When an agent reacts with a nanostructure, its texture, thickness, resistance, etc will change. These changes can be detected by ellipsometric measurements in which the ellipsometric parameters are determined. It is an object of the invention to use ellipsometry technique and equipment to determine change in texture, thickness, resistance when a nanostructure, especially when nanofilm reacts with an analyte/agent.
Electronic noses and electronic tongues: There are several gas sensors available on the markets among which are metal oxide sensors, often referred to as Tagushi sensors. They are composed of metal oxide(s) having a porous form, generally doped with a metal. They are operated at elevated temperatures of 100° C. to 600° C. in order to allow combustion of the analyte at the metal oxide surface, inducing a change of oxygen concentration and therefore a change in conductance. Metal oxide sensors are generally employed as single devices to detect toxic or flammable gases. If the oxide or other nanolayer undergoes an irreversible change in resistance and other properties when it reacts with an analyte, it can be used for monitoring total exposure to the analyte. When destroyable nanostructures are used as electronic noses and tongues, they can be used for monitoring degradation/spoilage of food, where the nanolayer is in direct contact with food (including above food but inside the package). The changes can be monitored visually if there is a change in color or transparency or with noncontact or contact analytical equipment.
Basic instrumentation: The detecting/monitoring systems proposed herein can also be composed of some basic subsystems, (1) Source/supplier unit: The source can be an electrical current, electromagnetic ionizing or non ionizing radiation (micro/radio waves, infrared, electron, gamma ray, neutron), gas and alike. Power source could be an AC or DC depending upon the device. (2) Cell: This could contain many components to support the nanostructure, (3) Detector/sensor: To monitor a change occurred in the cell/nanostructure, (4) Analyzer: Analytical technique or instrument, such as spectrophotometers (X-ray, visible, IR, microwave, FTIR, Raman spectroscopy), electrometer, etc, (5) Processor: A computer with the proper software to process the data and (6) Display: A monitor or printer to show the changes.
Capacitor: A capacitor, two parallel conductors separated by a dielectric, can be formed by rolling a metalized plastic film that includes a plastic film serving as a dielectric and two metal layers serving as electrodes. For a long life of a capacitor, the plastic film serving as a dielectric is selected from the group consisting of polyethylene terephthalate resin, polypropylene resin, polyethylene naphthalate resin, polycarbonate resin and the like. The metal serving as an electrode is selected from the group consisting of zinc (Zn), aluminum (Al), aluminum alloy and the like.

A capacitor of the present invention can be composed of two very thin reactive metal layers having a thickness in nanometers and a dielectric layer which has capability producing an activator when subjected to an analyte, such as electromagnetic radiation and magnetism. The destroyable capacitor for monitoring ionizing radiation can be composed of a very thin layer of radiation sensitive material, such as polyvinyledene chloride (PVDC) on a nano thin conductive layer or between nano thin metallized thin plastic films. There are many modifications of the capacitor. For example, a thin PVDC film can be metallized on both its sides. The destroyable capacitor can be rolled like other capacitors. In this case, the precursor film, such as that of PVDC will produce acids, such as HCl upon radiation. HCl will change the dielectric property of PVDC and/or can react with the thin metal layer and simultaneously change the resistance of the electrodes. Materials which undergo change in dielectric properties upon radiation can be used as a material for the dielectric layer that includes materials which undergo degradation, crosslinking, polymerization and formation radicals.

The capacitor can also be a nanocapcitor as well. The size of the components of the capacitor can be in form nano to any large desired. The destructible capacitors can be connected in a series or in a parallel or in a combination of them as needed.

Change in properties, such change in conductivity/resistance, voltage, current, capacitance, ability to hold charge and/or combination thereof can be used for monitoring action of an agent, such as radiation. The radiation dosimeter capacitor can be electrical/electrolytic double layer or ion type.

The destroyable capacitors can be used for monitoring anything which can diffuse or pass through the capacitor, especially electromagnetic ionizing or non-ionizing radiation from radar/radio (10³ meter to 10⁻¹² meter) wave to cosmic wave of mega and giga volt energy. Radiowave (10³ meter), microwave, IR, visible, UV, X-ray, gamma ray (0.1 Angstrom). Monitoring the radiation will depend upon the pre-cursor or activator used.
Piezo electric: The dosimeters can also be made by selecting piezoelectric nano materials which are sensitive to analyte and change the piezoelectric properties. According to the invention, the manufacturing process comprises the stacking of at least one destroyable piezoelectric element and of at least two metallic electrodes.
Filters for radiation dosimeters: The dosimeter device could be made of more than one dosimeter system, one having no filter while the others having filters, such as lead, cadmium, copper, boron etc of different thicknesses for selectively filtering of some radiation of certain energy.
Neutron: For monitoring neutrons the dielectric layer can contain compounds having a high neutron cross section, such as boron and lithium compounds which produce alpha particles when interact with a neutron.
Blood RAD/TTI: Certain perishables, such as fresh blood and some food are radiated. Once radiated, they have shelf life. These types of perishables need two indicators, one for indicating radiation exposure and the other for indicating shelf life. It is also possible to use two radiation dosimeters of different sensitivities for these types of perishables. The higher sensitivity will show a change upon radiation while the other will show radiation and shelf life. The device, having halocompounds as a precursor, can be used as for monitoring radiation and/or time-temperature. Radiation will produce an acid which will then etch the metal. As there is a delay, this is good for blood and other foods/perishables which are radiated and after radiation they have shelf life. The result of such radiation followed by the time-temperature indicator is shown in FIGS. 25(a) and 25(b) both are visual and measure the resistance of the nano thick layer of a metal.
High sensitivity dosimeter: The capacitor can be charged before radiation. When radiated, the charged electrode will produce a charged species which will degrade the destroyable dielectric layer. One can measure the dose either by measuring the charge, resistance of the nano dielectric layer and the nano metal layer or the capacitance of the capacitor.
Dielectric layer: The dielectric layer of the capacitor can be a destroyable polymer, such as PVDC containing halocompounds, such as chloroform or trichloroethane.
Autocatalytic: Production of an activator can be accelerated by an autocatalytic chain reaction, e.g., dehydrohalogenation of polymers, such as polyvinylchloride and polyvinylidene chloride and other halo compounds, such as 1,2,3,4,5,6-hexachlorocyclohexane and perchlorinated hydrocarbons.
Design: The dosimeter can be made in many different ways and can have many designs. The sensor could be disposable and electronic. It could be in the form of a badge or a table top unit. The holder can be similar to those available commercially, described in prior art and in patent application Ser. No. 12/478,232. The holder can be composed of an area to receive the element/sensor. The dosimeter can be inserted in a unit which can read properties, such as conductance, capacitance, charge, etc and read the dose from the calibration. Proper software and calibration can be developed and used for calculating the dose.
False signals: The dosimeter can be designed for monitoring false positives, false negatives, other undesirable effects of ambient conditions and tampering. The system can also include the devices and processes for the correction of the undesired effect of ambient conditions, such as time, temperature, time-temperature, shelf life, humidity, UV/sunlight, air pollutant and other undesirable ambient conditions.
Two sensors: Two sensors can be supplied to the users, one to be stored away from the source of users and the other for monitoring the background dose.
Methods of determination and standards: One can use ASTM methods for determination of change in properties. For example, change in volume or surface resistivity can be determined by ASTM D 991 and ASTM D 257, respectively.
Use of conducting polymers: Conducting/doped polymers, such as polyphenylenevinylene, polyacetylene, polythiophene, polypyrrole, and polyaniline and polyphenylene sulfide can be used for making the electrodes. Undoped conducting polymers containing halocompounds can be as a dielectric layer. Upon radiation, acids such as HCl, HF or iodine will increase the conductance of the layer.
Container/holder: The container for the dosimeter or sensor should preferably be opaque and impermeable to protect from UV light and other ambient conditions, such as impermeable to oxygen and water/humidity.
Medium: Dielectric layer/medium does not have to be solid. The medium can be liquid, gel, semisolid, gas, vapor or even a plasma state or mixture thereof. The medium can be an emulsion of a halo compound or a mixture thereof with water using preferably non-ionic surfactants. The medium can have one more additive to control the reaction, either to accelerate or retard. Water is a preferred additive, preferably in the form of a solution or emulsion. The dielectric layer can be composed of microemulsion and nanoemulsion
Thickness: The conductive layer can be a metal, an alloy, a conductive polymer or a mixture of conductive polymers. The thickness of the conductive layers and dielectric layer can be from a nano meter to microns or thicker. However, one of them should be thinner and preferably in the nanometers range.
Halocompounds: Examples of the halogenated organic compounds include halogenated hydrocarbons, halogenated alcohols, halogenated ketones, halogenated ethers, halogenated esters, halogenated amides, halogenated sulfones, halogenated phosphates, and halogenated heterocyclic compounds. In the halogen compound, two or more halogen atoms are preferably bound to one carbon atom. It is more preferred that three or more halogen atoms be bound to one carbon atom.

Examples of the halogenated hydrocarbons include carbon tetrabromide, iodoform, ethylene bromide, methylene bromide, amyl bromide, isoamyl bromide, amyl iodide, isobutylene bromide, butyl iodide, diphenylmethyl bromide, hexachloroethane, 1,2-dibromoethane, 1,1,2,2-tetrabromoethane, 1,2-dibromo-1,1,2-trichloroethane, 1,2,3-tribromopropane, 1-bromo-4-chlorobutane, 1,2,3,4-tetrabromobutane, tetrachlorocyclopropane, hexachloro-cyclopentane, dibromocyclohexane, and 1,1,1-trichloro-2,2-bis(4-chlorophenyl)ethane.

Examples of the halogenated alcohols include 2,2,2-trichloroethanol, tribromoethanol, 1,3-dichloro-2-propanol, 1,1,1-trichloro-2-propanol, di(iodohexamethylene)aminoisopropanol, tribromo-t-butyl alcohol, and 2,2,3-trichlorobutane-1,4-diol.

Examples of the halogenated ketones include 1,1-dichloroacetone, 1,3-dichloroacetone, hexachloroacetone, hexabromoacetone, 1,1,3,3-tetrachloroacetone, 1,1,1-trichloroacetone, 3,4-dibromo-2-butanone, 1,4-dichloro-2-butanone, and dibromocyclohexanone.

Examples of the halogenated ethers include 2-bromoethyl methyl ether, 2-bromoethyl ethyl ether, di(2-bromoethyl) ether, and 1,2-dichloroethyl ethyl ether.

Examples of the halogenated esters include bromoethyl acetate, ethyl trichloroacetate, trichloroethyl trichloroacetate, homopolymer or copolymer of 2,3-dibromopropyl acrylate, trichloroethyl dibromopropionate, and ethyl alpha, beta-dichloroacrylate.

Examples of the halogenated amides include chloro-acetamide, bromoacetamide, dichloroacetamide, trichloro-acetamide, tribromoacetamide, trichloroethyltrichloro-acetamide, 2-bromoisopropionamide, 2,2,2-trichloro-propionamide, N-chlorosuccinimide, and N-bromosuccinimide.

Examples of the halogenated sulfones include tri-bromomethyl phenyl sulfone, 4-nitrophenyl tribromomethyl sulfone, and 4-chlorophenyl tribromomethyl sulfone.

Examples of the halogenated phosphates include tris(2,3-dibromopropyl)phosphate.

Examples of the halogenated heterocyclic compound include 2,4-bis(trichloromethyl)-6-phenyltriazole.

Particularly preferred halogen compounds are tri-bromomethyl phenyl sulfone and 2,4-bis(trichloromethyl)-6-phenyltriazole.

Agricultural chemicals including, for example, ethyl-4-[4-(4-trifluoromethylphenoxy)phenoxy]-2-pentenoate, butyl-2-[4-(5-trifluoromethyl-2-pyridyloxy)phenoxy]propionate, N-benzyl-2-isopropylpivalamide, N,N-dialkyl-2-chloroacetamide, S-ethyl-N,N-diethyl carbamate, 4-octanoyloxy-3,5-dibromobenzonitrile, 2-chloro-2′,6′-diethyl-N-(n-propoxyethyl)-acetanilide, 2-(2-chlorobenzylthio)-5-propyl-1,3,4-oxadiazole, 2-(1,2-dimethylpropylamino)-4-ethylamino-6-methylthio-1,3,5-triazine, hexachloroacetone, tris-[2-(2,4-dichlorophenoxy)ethyl]-phosphite, and 2-(2-chlorophenyl)methyl-4,4-dimethyl-3-isooxazolidinone can also be used.

Preferred are the trihaloacetates wherein all the halogen atoms are the same and especially the trichloroacetates. Illustrative of the compounds which can be employed in the practice of the present invention are methyl trichloroacetate, ethyl tribromoacetate, isopropyl trifluoroacetate, tert-butyl triiodoacetate, n-octyl dibromochloroacetate, n-decyl dichlorofluoroacetate, 1-ethyl-1-n-propylheptyl chlorodiiodoacetate, n-pentadecyl trichloroacetate, n-eicosyl trichloroacetate, cyclopentyl trichloroacetate, cyclohexyl trichloroacetate, phenyl trichloroacetate, 1-naphthyl trichloroacetate, 2-naphthyl trichloroacetate, cyclopentylmethyl trichloroacetate, 7-cyclohexylheptyl trichloroacetate, benzyl trichloroacetate, 3,4-diphenylbutyl trichloroacetate, 2-methylcyclopentyl trichloroacetate, 3,4-di-n-butylcyclopentyl trichloroacetate, 2,3,4-tri-n-pentylcyclopentyl trichloroacetate, 4-methylcyclohexyl trichloroacetate, 2,4,6-triisopropylcyclohexyl trichloroacetate, 4-n-dodecylcyclohexyl trichloroacetate, 4-phenylcyclohexyl trichloroacetate, 4-tetradecylphenyl trichloroacetate, 4-methylphenyl trichloroacetate, 2,4,6-triethylphenyl trichloroacetate, 3,5-di-n-butylphenyl trichloroacetate, 4-cyclohexylphenyl trichloroacetate, and the like.
Pre-treated: The device can be pre-radiated to dissolve or to make the oxide layer thinner or by adding a controlled amount of an activator/additive, such as HCl or other etchant so it can be easily destroyed or thinned to a desired layer. In this case, once an oxide layer is destroyed water can react and dissolve the metal layer. A preferred activator/additive is one which gets adsorbed on the oxide layer (if it is there).
Protective layer: If required, in order to protect the metal layer from forming an oxide layer, it can be coated with a layer which is non permeable to oxygen and moisture or with another very thin layer of a metal, such as copper which can be easily destroyed.

### Conducting Polymers

The conductive layer can also be made from conductive ink or paint containing fine particles of a conductive material, such as metal or conductive polymer. The materials used for conducting inks include carbon, copper, silver, aluminum, silver-aluminum, indium tin oxide, fluorine doped tin oxide, as well as specialty materials, such as the copper indium gallium diselenide (CIGS) for the active layer in some PVs (photovoltaics).

Electrical conductivity can be induced in polymers selected from the group of substituted and unsubstituted polyanilines, polyparaphenylenvinyles, substituted and unsubstituted polythiophenes substituted and unsubstituted poly-p-phenylene sulfides, substituted polyfuranes, substituted polypyrroles, substituted polyselenophene, polyacetylenes formed from soluble precursors, combinations thereof and blends thereof with other polymers.

The polymers may contain a doping precursor, selected from the group of onium salts, iodonium salts, triflate salts, borate salts, tosylate salts and sulfonoxylimides. Conductivity can be selectively induced in the polymers by selectively doping upon selective exposure to a source of energy, such as electromagnetic radiation, e.g., an electron beam or X-ray.
Dopants: Dopant for making the polymers conductive may comprise one or more of: iodine, bromine, antimonypentafluride, phosphoruspentachloride, vanadiumoxytrifluride, silver(II) Fluoride, 2,1,3-benzoxadiazole-5-carboxylic acid, 2-(4-biphenylyl)-5-phenyl-1,3,4-oxadiazole, 2,5-bis-(4-aminophenyl)-1,3,4-oxadiazole, 2-(4-bromophenyl)-5-phenyl-1,3,4-oxadiazole, 4-chloro-7-chlorosulfonyl-2,1,3-benzoxadiazole, 2,5-diphenyl-1,3,4-oxadiazole, 5-(4-methoxyphenyl)-1,3,4-oxadiazole-2-thiol, 5-(4-methylphenyl)-1,3,4-oxadiazole-2-thiol, 5-phenyl-1,3,4-oxadiazole-2-thiol, 5-(4-pyridyl)-1,3,4-oxadiazole-2-thiol, methyl viologen dichloride hydrate, fullerene-C60, N-methylfulleropyrrolidine, N,N′-bis(3-methylphenyl)-N,N′-diphenylbenzidine, triethylamine, triethanolanime, trioctylamine, triphenylphosphine, trioctylphosphine, triethylphosphine, trinapthylphosphine, tetradimethylaminoethene, tris(diethylamino)phosphine, pentacene, tetracene, N,N′-Di-[(1-naphthyl)-N,N′-diphenyl]-1,1′-biphenyl)-4,4′-diamine, 4-(diphenylamino)benzaldehyde, di-p-tolylamine, 3-methyldiphenylamine, triphenylamine, tris[4-(diethylamino)phenyl]amine, tri-p-tolylamine, acradine orange base, 3,8-diamino-6-phenylphenanthridine, 4-(diphenylamino)benzaldehyde diphenylhydrazone, poly(9-vinylcarbazole), poly(1-vinylnaphthalene), triphenylphosphine, 4-carboxybutyl)triphenylphosphonium bromide, tetrabutylammonium benzoate, tetrabutylammonium hydroxide 30-hydrate, tetrabutylammonium triiodide, tetrabutylammonium bis-trifluoromethanesulfonimidate, tetraethylammonium trifluoromethanesulfonate, oleum, triflic acid and/or magic Acid. Dopants may be bonded covalently or noncovalently to the film. The film may have a stabilizer. The stabilizer may be a relatively weak reducer (electron donor) or oxidizer (electron acceptor). Additionally or alternatively, the stabilizer and dopant may comprise a Lewis base and Lewis acid.
Coating methods: In addition to methods, such as chemical vapor deposition, physical vapor deposition, laser assisted pyrolysis deposition, electron-beam physical vapor deposition and thermal spray, one can use spray-coating, dip-coating, drop-coating and/or casting, roll-coating, transfer-stamping, slot-die coating, curtain coating, [micro]gravure printing, flexoprinting and/or inkjet printing for making one or more layers required herein.
Substrate: Use of a substrate depends upon the device. The substrate can be flexible or rigid, and include, but not limited to, glass and/or plastics (e.g., polyethylene terephthalate (PET), polyethylene naphthalate (PEN), polycarbonate (PC) and/or polyethersulfone (PES)) or metals.
Humidity and relative humidity indicators: As shown in Example 4, using a nano layer of metal, we developed a humidity and relative humidity indicator by selecting acidic or basic compounds as activators which get dissolved when a certain relative humidity is reached, they get dissolved and etch/dissolve the activator/metal layer. Hygroscopic materials are ideal for monitoring total exposure as they will keep on dissolving the activator and etching/dissolving the metal layer. Once the oxide layer is dissolved, water has the capability of etching/reacting/dissolving certain metals, such as aluminum.

The layer for humidity and other indicators can be created by dispersing fine particles of activator, such as materials which etch/dissolve the indicator/metal layer in a polymer either by melt processing, UV curing etc and then laminating between an indicator tape and a protective film.

For detecting chemicals other than water/humidity, one needs a proper activator and nano indicator structure.
Dish washing indicators: Similarly, nanostructures of precursors, activators and indicators which have low reactivity of humidity or water can react at higher concentrations or higher temperatures and undergo measurable or noticeable color changes can be used for monitoring doneness of dish washing in a dish washer and the drying of clothing in a dryer.
Steam Sterilization indicators: Similarly, nanostructures of precursors, activators and indicators which have low or no reactivity to water and steam at lower concentrations or higher temperatures (below 100° C.) but react at higher concentrations or higher temperatures (e.g., saturated steam at 120° C. and above) and undergo measurable or noticeable color change can be used for monitoring steam sterilization.

Using pre-cursors disclosed in U.S. patent application Ser. No. 12/478,232, one can develop a sterilization indicator for ethylene oxide, oxidants such as hydrogen peroxide and perchloroacetic acid, plasma, dry heat, radiation and aldehydes such as formaldehyde. Instead of using a nano metal layer one can use proper color materials, such as dyes and pigments as indicators and appropriate activator or precursors.

### Nanoantenna and NanoRFID:

A nanoantenna is a device that absorbs a small wavelength of electromagnetic radiation through resonance. The nanoantennas are made of metal wires and spheres only about 10 nanometers thick—or roughly 100 atoms (or 5-100 nm) wide. They are an example of “left-handed” materials, meaning they are able to reverse the normal behavior of visible light and other forms of electromagnetic radiation.

We have demonstrated (U.S. patent application Ser. No. 12/478,232) that macro-size antenna and other electronic path ways can be created by masking a metallized plastic film followed by selective etching of the unmasked metal layer with an activator tape. Using the same technique, one can also create micro and nanoantennas. For making a nanoantenna, one can print the mask/resist nanolithography using techniques, such as imprint soft writing, dip pen, photo/laser and e-beam, soft, self assembly and micro-contact lithography.

A metallized plastic film can be selectively printed with nanolithography followed by etching or etched with a laser (e.g., by ablation) to make any shaped antennas. The antenna can be created in form of wings or lines, e.g., tiny square or other shaped spirals on the metallized plastic film. Etching can be done with gas, vapors, liquids or plasmas. Nanoantennas can absorb energy produced through the infrared spectrum. Infrared energy is produced in massive quantities by the sun, a portion of which is absorbed by the earth only to be released as radiation after the sun has set. These nanoantennas can absorb energy from both the rays of the daylight sun and the heat radiated from the earth at a higher efficiency than modern solar cells.

Though in principle any metal, metal alloy or conducting material can be used, the preferred metal is highly environmentally stable metal, such as silver or gold or their alloys. Plastic substrate can also be any but preferably dimensionally stable and treated to keep the metal antenna bonded to the plastic under harsh environmental conditions. The antennas preferably should be sandwiched between two films which do not absorb IR radiation.

The preferred metals are aluminum, gold, manganese, copper and their alloys. Under proper conditions, they can absorb most of the IR light.

The infrared rays create alternating currents in the nanoantennas that oscillate trillions of times per second, requiring a component called a rectifier to convert the alternating current to direct current. One needs nanorectifiers that go with our nanoantennas.

### Fabricating Nano-Optics

Nano-optic devices can be fabricated using semiconductor-like deposition, lithography, etching and coating processes. In general, a lithographic mask is prepared with the desired nanoscale features patterned on it. The original mask can be patterned using e-beam lithography, interference lithography or by combining multiple partial mappings and exposures to create spatial variations or arrayed optics.
Chemical dosimeters: There is a strong need for chemical dosimeters with high sensitivity in the parts per million (ppm) to parts per billion (ppb) level. Chemical dosimeters are needed for monitoring the total exposure to toxic agents, such as industrial chemicals and warfare chemicals. Nanomaterials, in general, have a very large surface area e.g., about 1600 m²/g. This large surface area translates into a large surface area available for the reaction and hence fast, high concentration and total exposure monitoring. The reaction or destruction of nanostructures can lead to a change in some specific properties of the device, for example, optical and electrical changes.

The etching technique can be used to destroy the nano item materials and devices by etching. Each material would be a different etchant depending upon the nature of the nanomaterials.

Devices having a destructible nanostructure can be used for monitoring warfare and bio-agents listed in our patent application Ser. No. 12/478,232.
Wedge shaped nanostructure: With a wedge shaped layer of nanostructures or step nanostructures one can continuously monitor and keep record of exposure.
Concentration and total exposure: Both stable (e.g., ZnO) and unstable (e.g., irreversibly reactive) nanostructure devices can be combined to monitor concentration and total exposure.
Reactive nanostructure: For dosimeter type monitoring system, one needs to use materials which react with an agent/analyte and are in the form of nanostructures.
Computer chips as dosimeters: One can use magnetic multilayers, for example, composed of sandwiches of cobalt, copper, and permalloy (nickel-iron), often called giant magnetoresistance (GMR) that change their electrical resistance when exposed to the magnetic field can be used as a dosimeter. The sandwich structures are known as spin valves, since they preferentially transmit electrons of one spin orientation. A related phenomenon is oscillatory magnetic coupling, an oscillation in the magnetic orientation of two layers with film thickness. If not protected, these metal layers can react with many chemicals and destroy the structures. Thus, by measuring the remaining bites, one can determine the total exposure. Likewise, iron oxide magnetic tape can be used.
Applications: Using the materials and processes disclosed herein, it is possible to create temporary, disposal and self destructive electronic devices once activated with an activator layer.
Advantages: The dosimeters disclosed herein will be inexpensive and can be incorporated in a personal ID.
Virus detection: A virus can also be detected by coating destroyable nanostructures on a substrate/electrode which have the capability of getting attached to a virus. These nanostructures can be self destructing and hence a change in their properties can be monitored. Viruses can also be monitored by passing a sample of air through a dispersion of nanostructures in a medium, such as water. When nanostructures adsorb/attach to a virus, they may react and undergo a change in their properties.
Monitoring combustible gases: Devices with destructible nanostructures can be used as dosimeters for monitoring combustible gases, such as carbon monoxide, oxygen, hydrocarbon, organic solvents and hydrogen sulfide. These devices can also be used for monitoring gases/burned products produced during a fire.
Wireless communication: The results, data of the devices disclosed herein, can be sent by wireless communication.
Quantity required: A square centimeter of a 100 Angstroms thick layer of a metal, such as aluminum is about 1×10⁻⁷ mole or 6.029×10¹⁶ or 4.59×10¹⁶ atoms. 1 mm×1 mm area will be 1×10⁻⁹ mole. It will weigh about 1/1000^{th} of a milligram. Thus, an agent can be detected in ppm and ppb.
De-agglomeration: Nanostructures usually have a tendency to form clumps (“agglomerates”). One can use a dispersant/surfactant, such as ammonium citrate (aqueous) and imidazoline or oleyl alcohol (non-aqueous) for de-agglomeration or to modify the surface of the nanostructures.
Property changes & instruments: Conducting and semi-conductive nanostructures may undergo a change in electrical, ferroelectrical, dielectrical, magnetic, optical, quantum confinement, semi-conducting, surface plasmon resonance, brittleness, malleability, ductility and other properties. Instruments which can monitor these other properties mentioned herein can be used for quantitative analysis.
Etching for creation of nanostructures: Nanostructures can also be created by gas, vapor, plasma and liquid etching. The dry/plasma etching reported in the literature can be used, for example, with the plasma of oxygen and carbon tetrafluoride. The etchants or activators reported in our patent application can also be used.

Depending upon the material selected and the technique used for etching, one can create a variety of nanostructures including nano and quantum dots, tubes, wells and quantum wires.
Creation or increasing an oxide layer: Oxide and other layers, such as sulfate/ phosphates, can be created or the thickness of an existing oxide layer can be increased by oxidation with an oxidizing agent or by anodizing metallized/aluminized plastic film with a thick aluminum layer for increasing the induction period. The oxide layer then can be etched to create nanostructures.
Resistance of quantum wire: A quantum wire is an electrically conducting wire in which quantum effects are affecting transport properties. In a quantum wire, the classical formula for calculating the electrical resistivity of a wire (R=ρl/A, where ρ is the resistivity, l is the length, and A is the cross-sectional area of the wire) is not valid.
Metallized plastic film & aspect ratio: The aspect ratio (width/surface area divided by height/thickness) is incredibly high and essentially infinite for nano-film, such as a metallized plastic film.
Selective metallization and demetallization: Selective metallization can be achieved by selective etching/demetallization of unmasked areas, by printing a deposition-resistant material prior to metallization such as a vacuum pump oil on which metal does not deposit during vacuum deposition and metallization through a mask.

A selectively demetallized metal film is provided in which the metal film has different amounts of metal removed in different areas to provide a film having a graduated optical density from one area to another for a variety of applications. The amount of metal present in the film can vary gradually, continuously or in stages resulting in a series of bands or patches.

### Quantum Dots

Particle in a box: In materials where strong chemical bonding is present, delocalization of valence electrons can be extensive. The extent of delocalization can vary with the size of the system. Structure also changes with size. As size decreases (de Broglie wavelength) electrons (and holes) are confined (“particle in a box”). Electron-hole pair (excitons), due to a much longer wavelength of excitons in a semiconductor (1 micrometer compared to 0.5 nanometer for a metal) size confinement appears for N=10,000 atoms. Hence, as the size of a larger nanostructure decreases, e.g., by etching, electrons and holes will be confined in the reduced sized nanostructure and one can see a dramatic change in properties.

Semiconductor nanostructures are known for their photoluminescent and electroluminescent properties. Quantum dots (QDs) that can be used for the devices and processes herein are inorganic semiconductor nanocrystals having a typical diameter between 1-10 nm that possess unique luminescent properties. They are generally composed of atoms from groups II and VI elements (e.g. CdSe and CdTe) or groups III and V elements (e.g. InP and InAs) of the periodic table. The most commonly used QD system is the inner semiconductor core of CdSe coated with the outer shell of ZnS. The ZnS shell is responsible for the chemical and optical stability of the CdSe core. QDs can be made to emit fluorescent light in the ultraviolet to infrared spectrum just by varying their size. Quantum dots typically contain a charge somewhere between a single electron and a few thousand electrons.

Fundamentally, QD nanocrystals are fluorophores—substances that absorb photons of light, then re-emit photons at a different wavelength. Compared to traditional organic fluorophores used for fluorescence labelling in biological experiments, inorganic QDs have wider applications due to their high resistance to photobleaching, which enables visualization of the biological material for a longer time. Fluorophores are highly sensitive to their local environment and can undergo photobleaching, an irreversible photooxidation process which makes them non-fluorescent. Fluorophores can be optically excited only within a narrow range of wavelengths. Fluorescent emission is also restricted to a certain range of wavelengths whereas QDs can be excited with a single light source having wavelength shorter than the wavelength of fluorescence. Their fluorescent lifetime is higher (still measured in nanoseconds, though); and their photobleaching is reduced.

When a thin coating of a semiconducting material having the capability of forming a QD is etched with a proper etchant, it will form a QD at one stage before it gets further etched and destroyed. Thus, there will be a significant change in appearance and disappearance of fluorescence while forming a QD and destroying a QD during the etching process. Unless stabilized QDs have a high reactivity to ambient conditions. Even when stabilized with materials, such as ZnS, they still can be made to react with ambient conditions and the environment by destroying ZnS coating in situ or by using other permeable coating materials. Hence, they can be used for monitoring most of the processes and materials listed herein and in our patent application Ser. No. 12/478,232. The changes can be monitored with many techniques listed herein including change in fluorescence.

Destructible nanostructures can also be created by evaporate materials, such as metals on a porous substrate having nanoholes or dipping in a solution or liquid.

Liquid nanocrystals can be used for doping other nanostructure by their diffusion in other nanostructures. Thus, it can be easier to make p and n type devices.

If the nanocrystals adsorb oxygen and carbon dioxide reversibly, e.g., those made from perfluorocompounds, they can be used as synthetic blood for supply of oxygen.

Reactive nanostructures can be used for a rapid removal of toxic materials.
Monitoring radiation with QDs: QD are basically unstable unless stabilized with a core of a stabilizer, such as ZnS. QDs can be coated with precursors for monitoring radiation. Precursor coated QDs may undergo a significant change in fluorescence when exposed to high energy radiation, such as X-ray, gamma ray, electrons, neutrons, protons and alpha particles. The changes may even depend on energy and dose rate. Stability to ionizing radiation can be adjusted by selecting a proper stabilizer material and by the nature and coating thickness of stabilizers, such as ZnS. Stabilizers, such as precursors and activators, can be used which will stabilize the QDs but may become sensitive to ionizing radiation and other effects listed herein. One can also stabilize QDs by using phosphors. Use phosphors to emit UV visible light which then can excite the QDs.

Using the same principle one can also monitor other analytes, organic, inorganic, organometallic and biological agents.
Simultaneous changes in properties: As a nanostructure is being destroyed, there may be a simultaneous change in more than one property. Some properties may increase while others may decrease. E.g., when a thin film of aluminum is dissolved its transparency and resistance increase, in other words its opacity and conductance decrease. At the final stage of destruction/conversion (e.g., the last 1 nm or the last one atom/molecule), the transparency changes slowly but the electrical resistance goes up rapidly. Thus, the change in properties can be similar or disproportional.

There may be a change in the nature of a nanostructure when it is being destroyed, i.e., converted to another compound. One may destroy a nanofilm (e.g., 10 nm thick layer of aluminum layer) and in doing so, one may form nanorods and/or nanodots. It is not necessary that the product be nanostructure.

More than one property can be measured simultaneously as the nanostructure is being destroyed and a relationship can be developed between them. For example, a change in conductance and capacitance, in the case of a capacitor based radiation dosimeter, will change and can be measured simultaneously with an electrometer. Thus, the dosimeter devices proposed here will be more accurate and reliable.

Nanostructures are often referred to as substrate and its reaction product as product herein.
Change in plasmons: Plasmons, collective oscillations of conduction electrons, determine the optical properties of metallic nanostructures. The plasmon resonance in nanoparticles is determined not only by the nature of the metal or alloy that the particle is made of, but also by the size and shape of the particles. Due to their small size, the correlation of the shape and optical properties of individual nanocrystals is not straight forward. A dosimeter based change in plasmons can also be made and can be accurate.
Mixture of different types/nature of nanostructures: A mixture of properly selected nanostructures made from different materials, properties and nature can be used for making the dosimeters. The mixture could be essentially any mixture of two or more materials, for example, two different metals/semimetals/non-metals, metal and nonmetal, a metal and semimetal, semi-metal and nonmetal and organic and inorganic. For example, a mixture of nanostructures of copper and gold may undergo diffusion to form an alloy. A variety of devices, including dosimeters, can be made from the mixture of nanostructures of two different materials for some unique and unexpected properties.
Surface treatment, nucleation and growth of crystals: The surface of the substrate for metallized plastic film can be pre-treated, e.g., chemically or physically, e.g., etched or plasma treated before metallization to control formation of nuclei and their growth. The deposition could be at any angle, direct (90°) or angular, rate of deposition, temperature of deposition etc. The preferred metal layer is amorphous or having very small crystals. Other materials, such as a semiconductor, should preferably be crystalline.
Radiation: Metals, such as aluminum often have a thin layer of their oxide on their surface. Either the exposure to oxygen and humidity is minimized after the metallization or it should be removed by adding a chemical in the formulation which reacts with the oxide layer. The thinner the oxide layer, the more sensitive is the device. The device can be made oxygen free and sufficient quantity of an etchant is added to dissolve the oxide layer but not the metal. In such a case, water can dissolve some metals like aluminum if present in the formulation.

Depending upon the coating, one can measure change in many parameters, such as fluorescence, color, capacitance and resistance upon radiation to determine the exposure. The user can see a high dose from a change in opacity of the coating and monitor low and any dose accurately by measuring resistance, transparency or other sensitive methods including those mentioned herein. The device can be made to undergo a color change, if a dye which reacts with activator is produced upon radiation or with by products, such as metal salts.

Halo-compounds, such as 1,1,1-trichloroethane, are known to react with aluminum once the oxide layer is destroyed. Hence, once the oxide layer is destroyed, halo materials, such as carbon tetrachloride may react with the metal.

The metal could be any other metal than aluminum which is not affected by water so the linearity with dose can be obtained.
Nucleation and creation of nanostructures by etching: High density of nanostructures can be created by etching if there is a high density of nucleation during the metallization. High density of nucleation can be obtained by several methods, e.g., by preventing the nuclei formed from growing too large (i.e., controlled growth), for example, by rapid cooling of the metal vapor when it hits the substrate.
Additives: Activators, precursors, binders and additives and other compounds/formulations listed in our patent application Ser. No. 12/478,232 can be added to enhance the sensitivity of the devices and procedures disclosed herein.
Mixture of nanostructures: Dosimeter devices can also be created by the deposition of nanostructures of different shapes and materials, such as different metals, alloys, semiconductors, oxides and alike. These types of structures can be created by evaporating materials onto a substrate. The layers could be one metal on to the other or similarly more than one metal or alloy, a metal/semi-conductor/metal. The layer can be transparent or opaque and can also be oriented in different directions.
Determination zone: When nanostructures are etched, the change in many properties, such as electric resistance, is incredibly high as the particle size gets reduced to zero. The major change occurs when the size of the crystals is reduced from nano to a few atoms or molecules and then to essentially nothing. The zone for determination of the change in property is narrow, where the maximum change occurs. Hence, the devices based on the destruction of nanostructures should be highly sensitive, probably amongst the most sensitive.

Though we determined change in resistance, we expect that similar changes are expected with most of the other properties and analytical techniques listed herein and reported in the literature which can be used. For example, change in transparency is reported in patent application Ser. No. 12/478,232.
Electromagnetic radiation (X-ray) film: X-ray film can be made by coating halocompounds on a thinly metallized film or a mixture of nanostructures and halocompounds coated on a substrate. The coating formulation may contain a dye if a color change is needed. When exposed to ionizing radiation, the halocompounds will produce an acid which can etch the layer. Such films can undergo a change in transparency or a color change. If semi-transparent metallized film is used, the change can be gradual rather than having long induction period.
Nanostructures & changes: Etching or reduction in size of nanostructures can lead to a variety of changes. The size dependant properties of nanostructure include changes in physical, chemical, biological, pharmaceutical, toxicological, mechanical, nuclear, electrical, electronic, optical, thermal, quantum, magnetic, electromagnetic, ferroelectric, magnetotransport, excitation, super conductivity, crystal structure, crystallinity, transitions from one property to other, e.g., conductivity to super conductivity, color, luster, malleability, ductility, resistance, hardness, melting/freezing point, boiling point, density and other properties. The other properties include, absorption of electromagnetic radiation, acoustic, adsorption, attraction, band gap, catalytic activity, chirality, columbic, density, desorption, diffusion, electrical resistance, electron spin, freezing, hardness, interaction with electromagnetic radiation, ionic, melting, odor, phase change, plasma, pressure, reactivity, reaction rate and reaction mechanism, reflectance, refractance, repulsion, size, specific heat, solubility, specific heat, spectra, (new peak may appear and grow while old one may disappear), sublimation, surface area, surface reactivity, surface tension, thermal conductivity, photoconductivity, test, thermodynamic, transmittance and viscosity/flowability. These changes can be measured for the devices and processes listed herein and in our patent application Ser. No. 12/478,232 using the techniques and instruments listed herein. The devices and sensor can be in solid (e.g., a solid coating), semisolid, liquid, solution, gel and gas.
Nanostructures of materials which are radiation sensitive. Many materials are inherently radiation sensitive, e.g., halocompounds and radiochromic dyes. Their radiation sensitivity may change and their properties also may change upon radiation. Another example is a change in fluorescence. One can create a coating or film of such radiation sensitive nanostructures or a mixture of nanostructures of a metal, alloy or other high atomic number compounds (e.g. salts of barium) can be used to make them more sensitive to X-ray. A mixture of nanostructures of semi-conducting materials and halocompounds can be used for monitoring radiation.
CCD and radiation: Charge couple devices (CCD) made from materials which are less stable to ionizing radiation can be used for monitoring radiation.
Creating sub-nano structures by reacting nanostructures at their surface with an activator: The activator can be an etchant. Etching a thin layer of metal or other nano materials is one of the processes of making and then observing and determining properties of nanostructures. If the nanostructures change color during etching, they can be seen visually, e.g., metals, such as aluminum go from shiny white to gray to clear and simultaneously change in conductance. The change from silvery shiny white to gray indicates that the nano film is converted to nearly nanodots.

By this type of etching and other methods it is also possible to create subnanostructures, such as quantum dots and ultimately destroy the nanostructures/quantum dots of metals and semiconductors. Provided are methods of creating subnanostructures, such as quantum dots from nanostructures on a substrate or a layer of an electrode, such as gold.

Once a subnanostructure, such as a quantum dot is created on a substrate, it can be used for many applications, such as creating solar cell, LED and many others.

Typically the nano layer is on a dielectric substrate. If the substrate also has a metal which is not etched by the etchant, e.g., a gold layer, one can create subnanostructures, such as quantum dots directly on a gold electrode.

The quantum or nano dots so created can be of any other proper materials.
Scintillation and other fluorescence for radiation devices: Physical and chemical phenomena that can be used for the measurement of radiation includes ionization of atoms and molecules, excitation of atoms and molecules, scintillation, fluorescence, thermoluminescence (TL), damage of the solid state induced chemical reactions and scintillation.
Nano-OSL: Nanostructures can be prepared from properly doped organic, organometallic and inorganic materials to make nanoOSL (nano-Optically simulated Luminescence) and nanoTLD (Nano-ThermoLuminescence Dosimeter). Upon irradiation, electrons can get trapped between the valence and electron band of such nanostructures. The ionizing radiation can produce electron-hole pairs—electrons being in the conductance band and holes in the valance band. The electrons which have been excited to the conduction band may become entrapped in the electron or hole traps. In the case of OSL (Optically Simulated Luminescence) dosimetry, under stimulation of light, the electrons may free themselves from the trap and get into the conduction band. From the conduction band they may recombine with holes trapped in hole traps. If the center with the hole is a luminescence center (radiative recombination center) emission of light will occur. The photons can be detected/imaged using devices, such as a photomultiplier tube and CCD camera. The signal from the detecting system is then used to calculate the dose that the material had absorbed.

If the NanoOSL material is destroyable nano-OSL (i.e., loses its OSL properties), the process will be irreversible and the dose can be recorded from the remaining destroyable materials.

NanoOSL and other radiation sensitive devices can be used for measurement of radiation dose in the tissues of health care, nuclear, research and other workers.

Materials from which OSL nanostructures can be prepared and methods that can be used for estimation of dosimeters are described in literature, for example “Optically Stimulated Luminescence Dosimetry” L. Boetter-Jensen, S. W. S. McKeever, and A. G. Wintle, ISBN-13: 978-0-444-50684-9, ISBN-10: 0-444-50684-5, ELSEVIER, 2003.
NanoTLD: Nanostructures can be prepared from properly doped organic, organometallic and inorganic materials, especially materials, such as calcium fluoride and lithium fluoride. A thin layer of such materials can be doped or etched to introduce defects. High energy radiation can interact with the crystal. It causes electrons in the crystal's atoms to jump to higher energy states, where they stay trapped due to impurities (usually manganese or magnesium in the crystal, until heated). Heating the nano-structure can cause the electrons to drop back to their ground state, releasing a photon of energy equal to the energy difference between the trap state and the ground state. Like nanoOSL, the nanoTLD can be used both for environmental monitoring and for staff personnel in facilities involving radiation exposure, among other applications.

By selecting proper inorganic materials made from lithium-8 and boron-12 with a high cross sectional area nanoOSL and nanoTLD can be made much more sensitive to neutrons. If the NanoTLD material is destroyable, nano-TLD (i.e., loses its thermoluminescence properties), the process will be irreversible and the dose can be recorded from the remaining destroyable materials.

Nanostructure TLD and OSL can be much more sensitive and stable by selecting proper materials and dopant.

NanoOSL and NanoTLD devices can be of any shape and size, including micro-dosimeter and film.

Semiconducting nanostructures, e.g., that of Ge, Si, Ge(Li) and Si(Li) can be used for monitoring radiation.
Applicability to our past patent application: The nanodevices and associated methods disclosed herein can also be used for making monitoring devices and processes (such as time, temperature, time-temperature, freeze, thaw, humidity, doneness of foods with microwave, sterilization indicators) disclosed in our patent application Ser. No. 12/478,232 can also be created by destruction of nano-structures and processes disclosed herein. It is not necessary that these devices can be created only by two dimensional nanostructures.
Destructible Metals used to make indicators: Typically, aluminum and copper in high purity are used to make RFID antennas and other electronic circuits for environmental stability. Certain thickness is required, e.g., 5-15 microns for RFID antennas. Higher the thickness, difficult it is to etch by weak acids and based. However, certain metal alloys such aluminum and indium which more vulnerable to humidity and other chemicals, especially salts, acids and bases. Similarly, these electronic circuitries can be made of metals which can be easily attacked by activators such as oxygen, water, non toxic and/or hazardous compounds, such as salts, acids and bases. Thus, if the conductor is destroyed, the electronic device will not function, may function improperly, but in a predictable way. These conductive paths will be thicker to perform the job but easily destroyable. The electronic devices made from destructive or vulnerable materials can be used. These devices, using reactive metals or metals alloys, can be under inert atmosphere. These highly reactive alloys can be used for making other indicating devices as well.
Induction period and processes of coating of Al₂O₃: The induction period of the indicating devices disclosed our U.S. patent application Ser. No. 12/478,232 is due to the slow etching of the oxide layer, such as aluminum oxide. Those indicating devices can also be created by intentionally creating such an oxide layer on any indicating layer other than metals. Oxides, such as that of aluminum (Al₂O₃) can be coated using processes reported in the literature for the devices disclosed herein and requiring a coating of oxide layer and/or other inorganic coatings. Sputter coating and other methods are in the process of conversion of evaporated aluminum to aluminum oxide. Aluminum oxide can also be created by vacuum evaporation of aluminum under controlled atmosphere of oxygen. When an activator/etchant destroys the oxide layer, it can change the color or transparency of the indicating layer.

Instead of using microencapsulated activators for activation of the indicating devices disclosed in our U.S. patent application Ser. No. 12/478,232, one can use nanotubes filled with an activator or precursor. When subjected to a process, they will produce or release an etchant/activator which will dissolve the metal or the indicator layer.
Nanotubes and Freeze indicator: Nano tubes can be filled with an aqueous solution of a dye. When frozen, they will rupture the nanotubes and the liquid will come out. One set of nanotubes can be filled with a colorless pH dye and the other with an acid or base and when they are frozen, they will rupture and a color change will occur. The rate of reaction can be controlled by a binder. Nanotubes can be that of an oxide, such as zinc oxide.

Upon freezing nano particles can aggregate. A simple example is where gold nanoparticles are modified with cysteine to make them selective for Cu(II) in solution. The presence of Cu(II) causes the nanoparticles to aggregate with a concomitant change in color from red to blue.
Need for highly sensitive methods: There is always a need for highly sensitive and selective devices for monitoring materials and processes. The sensitivity of a method depends upon the property used or monitored. A number of properties and the magnitude of change in those properties are incredibly high when nanostructures are reduced in size to nanodots or completely destroyed. One can essentially create nanostructures of any solid material.
Metal or colored materials as monitor: When the nanostructure monitors are metals, semimetals or other colored materials, such as a dye or pigment, delocalization of valence electrons can be extensive. The extent of delocalization can vary with the size of the system. Change in the size and delocalization/excitons can lead to different physical and chemical properties, such as optical properties, band-gap, melting point, specific heat, surface reactivity and many more listed herein.
Semi-conductors as indicators: Band-gap is the energy needed to promote an electron from the valence band to the conduction band. The band-gap changes with size when semiconductors, such as ZnO, CdS, and Si, are used as monitors. When the band-gaps lie in the visible spectrum, changing band-gap with size means a change in color and other optical properties. In a classical sense, color is caused by the partial absorption of light by electrons in matter, resulting in the visibility of the complementary part of the light. On a smooth metal surface, light is totally reflected by the high density of the electron's no color, just a minor-like appearance. Small particles absorb and lead to some color. This is a size dependent property. For example, gold, which readily forms nanoparticles but is not easily oxidized, exhibits different colors depending on particle size. Gold colloids have been used since early days of glass making to color glasses. Ruby-glass contains finely dispersed gold-colloids. Silver and copper also give attractive colors. Protected nano particles of certain metals, dyes and pigments can be used for making solventless printing inks and printing fabrics.
Nanostructures of dyes and pigments: Nanostructures of dyes and pigments can be of different colors than that of bulk. Dosimeters and other devices can be developed from these nano-colored materials. Some of these nanostructures can be liquid. They can be used where dyes and pigments are used, including very sensitive indicators, detectors and dosimeters. When such nanostructures are subjected to a treatment, they may undergo a color change.

Many solids will become liquid, semi-fluid or have flowing properties when they are in nano size. Dyes, pigments, their intermediate or reactants and moderators can be liquid and colorless or of different color in nano form. Liquid nanos can be stabilized with surface active agents/surfactants. These can be used for printing while minimizing pollution. The process of printing paper and fabric can be pollution free and can save energy. Different colors and shades can be obtained by proper mixing.
Printing and imaging: A substrate coated with nanostructures or nanodot and activators or precursors can be used for a large number of printing and imaging related products. If the coating undergoes an irreversible color change, e.g., white to black or vice versa upon melting of a nanostructure, it can be used for direct thermal printing. If it changes with radiation, such as UV light or X-ray, it can be used for printing and imaging. If it changes with ultrasonic radiation, it can be used for imaging and printing with ultrasound and monitoring ultrasound. If the substrate is clothing, it can be used for dyeing, i.e., printing fabric. Nanostructures which change noticeable colors with electrostatic forces can be used for Xerox type printing.
Nano-electrochromic materials: Nano-sized destroyable electrochromics can be used for monitoring one or more of the processes and materials disclosed herein. Nanostructures made from electrochromic materials can also be used as a dosimeter as they will also change in their properties when exposed to analyte.
Nano-thermochromic materials and temperature indicators: Thermochromic nanostructures may undergo a color change when a certain temperature is reached. Many nanostructure metals, alloys, semiconductors and other colored or opaque materials have a lower melting point. If they are heated above their melting point they will undergo coalescence/fusion and thereby lose their nanostructure properties, including a change in color and opacity/transparency. The temperature for the change can be varied by adding proper reactive and non-reactive additives, especially surface active agent and polymers or other nano-structures. These thermochromic nanostructures include nano-liquid crystals. These thermochromic nanostructures can be used where normal thermochromic materials are/can be used.

The thermochromism of the nano-thermochromic materials can be reversible, irreversible or in between.
Photochromic nanostructures: There are a large number of organic and inorganic reversible and irreversible photochromic materials reported in literature and used. Reversible photochromic nanostructures are normally more stable but their nanostructures may not be that stable. Nanostructures of irreversible and reactive reversible materials also can be used as dosimeters.
Radiochromic nanostructures: Like irreversible photochromic nanostructures, radiochromic nanostructures can be prepared. They will undergo color and other change in properties when radiated with ionizing radiations, such as UV, X-ray, gamma ray, electrons, protons and neutrons. These materials can be used for one, two and three dimensional dosimetry. Examples of materials that can be used for radiochromic nanostructures are reported and are given in this and our patent application Ser. No. 12/478,232. If the change is reversible, it can be used for monitoring energy, the type of radiation and the dose rate.
Change in surface tension: Surfaces of plastic films and metals are not wettable with water. During the etching of a metallized/aluminum plastic film with phosphoric acid, we observed that the etched surface becomes increasing wettable with water as the film becomes grayish or nearly transparent. The results indicate that the surface energy increases substantially as the particles size decreases below about 3 nm.
Magnetic monitors: When magnetic materials, such as Fe, Co, Ni, and Fe₃O₄, are used as monitors, magnetic properties are also size dependent. The ‘coercive force’ (or magnetic memory) needed to reverse an internal magnetic field within the particle is size dependent. The strength of a particle's internal magnetic field can be size dependent. When the nanostructures of these magnetic materials are attacked by an agent, their magnetic properties will change and hence can be used as monitoring agents/analytes.
Electrical resistance and size: For metals, conductivity is based on their band structure. If the conduction band is only partially occupied by electrons, they can move in all directions without resistance (provided there is a perfect metallic crystal lattice). Electric current is a collective motion of electrons in a bulk metal and Ohm's law: V=RI is valid. Band structure begins to change when metal particles become small. Discrete energy levels begin to dominate and Ohm's law is no longer valid. If a bulk metal is made thinner and thinner, until the electrons can move only in two dimensions (instead of 3), then it is a 2D quantum confinement. The next level is a quantum wire and ultimately a quantum dot. Thus, one can expect a dramatic change in properties when three or two dimensional nanostructures are gradually destroyed, e.g., by etching.
Shape of nanostructure: The nanostructure can also be (1) a cluster, a collection of units (atoms or reactive molecules), e.g., up to about 50 units, (2) colloids, a stable liquid phase containing particles in the 1-1000 nm range, (3) a colloid particle is one such 1-1000 nm particle, (4) a nanoparticles, a solid particle in the 1-100 nm range that could be noncrystalline, an aggregate of crystallites or a single crystallite and (5) nanocrystal, a solid particle that is a single crystal in the nanometer range.
Adsorption/catalysis: Adsorption is like absorption except the adsorbed material is held near the surface rather than inside. In bulk solids, all molecules are surrounded by and bound to neighboring atoms and forces are in balance. Surface atoms are bound only on one side, leaving unbalanced atomic and molecular forces on the surface. These forces (Van der Waals force, physical adsorption or physisorption) attract gases and molecules.

Surface chemistry is important in catalysis and detection of materials. Nanostructured materials have some advantages, e.g., huge surface area, high proportion of atoms on the surface. Enhanced intrinsic chemical activity as size gets smaller is likely due to changes in crystal shape. For example, when the shape changes from cubic to polyhedral, the number of edges and corner sites goes up significantly. As the crystal size gets smaller, anion/cation vacancies can increase, thus affecting surface energy; also surface atoms can be distorted in their bonding patterns.

Hence, if an analyte reacts or destructs a nanostructure, the molecules of the analyte will readily react and its exposure can be monitored by a rapid change in the properties of the nanostructure.

The advantages of nanoparticle catalysts are very large surface area, enhanced intrinsic chemical reactivity, edge and corner effect, anion/cation vacancies, distorted in bonding patterns. Examples of catalyst materials are Pt (or Pd), Au based, other metals (Cu, V, Rh), nonmetallic: MgO, MoS₂, CeO₂₋ₓ, NiO, Cr₂O₃, Fe₂O₃, Fe₃O₄, Co₃O₄, and β-Bi₂Mo₂O₉. Examples of homogeneous catalysts are acids, bases and capped nanoparticles and those of heterogeneous catalysts and dispersed on highly porous support are porous silica, titania, alumina, zeolites. Nanocatalysts can be used for conversion of an analyte into an activator which then can react with a nanostructure. The change in catalytic activity with an analyte can be used for monitoring analytes.
Particle sizing techniques: Several methods, such as sieve size analysis, sedimentation, laser diffraction light, scattering, dynamic light scattering and photon correlation, spectroscopy, light obscuration, electrozone sensing, microscopy/image analysis, electroacoustic, acoustic attenuation and field flow fractionation can be used for determination of the nanomonitor/nanostructures and changes in them when an analyte reacts.
Surface modifier/stabilizer: The monitoring system made from nanostructures may contain surface modifiers/stabilizers, such as surfactants, coupling agents (silanes) and polymers, such as natural polymers (such as gelatin, agar, cellulose acetate, cellulose nitrate, cyclodextrins) synthetic polymers (such as vinyl polymers with polar side groups, such as polyvinylpyrrolidone, polyvinyl alcohol, vinyl pyrrolidone—vinyl alcohol copolymer, poly electrolytes), long-chain alkylammonium cations and surfactants, sulfonated triphenylphosphine and alkanethiol.
Silanes: Silanes that can be used for stabilization of nanostructures of nanomonitors includes compounds containing silicon-hydrogen bonds, SiH₄, trichlorosilane: HSiCl₃, disilane: H₃SiSiH₃, methylsilane:CH₃SiH₃, methyldichlorosilane:CH₃SiHCl₂, triethylsilane: (C₂H₅)₃SiH, thiol: sulfur analogous of alcohol, mercaptan, 2-mercaptoethanol: HSCH₂CH₂OH, mercaptoacetic acid: HSCH₂COOH, 1-amino-2-propanethiol: H₂NCH₂CH(SH)CH₃, thiophenol: C₆H₅SH and dithiol: 1,2-ethanedithiol: HSCH₂CH₂SH.
Freeze indicator: Nanostructures and materials which undergo phase separation when frozen or react with a material which is phase separated upon frozen, such as those described in U.S. Pat. No. 6,472,214 can be used as freeze indicators. Nanostructures which go from clear to opaque or vice versa, undergo a color change when frozen or undergo a visual or measurable change in chemical or physical properties can be used as freeze indicators. If the frozen system further undergoes change, e.g., color change upon thawing then it can be used as a thaw or TTI indicator as well. If an etchant or its solution phase separates when frozen and etches the metal layer or fine metal particles it can be used as freeze indicator.
Thaw indicator: Stabilized nanostructures may remain stable under ambient conditions, such as room temperature but may become unstable if frozen and may undergo a color or change in fluorescence. When such a frozen system is thawed it may undergo a color change or other changes. Such systems can be used as thaw indicators.
Dry heat sterilization indicators: Nanostructures having a melting point at higher temperatures, e.g., 160° C. (used for sterilization) can also undergo several changes, including color changes due to melting and the formation of larger structures. Such systems, organic, inorganic or otherwise can be used as dry heat indicators.
Ethylene oxide sterilization indicators: Nanostructures which react, or systems composed of nanostructures and a precursor which produce an activator when reacts with ethylene oxide and hydrogen peroxide or other oxidants and undergo measurable or noticeable (e.g., color) change, can be used for monitoring sterilization with them. These systems are disclosed in our patent application Ser. No. 12/478,232 for metallized plastic film and micron sized metal particles. The system can also be used for monitoring a low level of ethylene oxide gas. By selecting a proper pre-cursor, one can monitor other toxic agents using the methods and equipment described herein.
Alcohol indicator: If a nanostructure or a mixture of nanostructures and an activator/precursor is sensitive to alcohol, it can be used for monitoring alcohol, e.g., in breath and similar other chemicals.
pH indicator: If a nanostructure or a mixture of a nanostructure and an activator/precursor is sensitive to pH (acids or base, H⁺ or OH⁻), it can be used as a pH indicator or monitoring acids, bases and their strength.
Embedded in a binder: The nanostructures of the systems described herein can also be dispersed in a polymeric binder. The binder may change the properties and behavior of the nanostructures.
Nanolithography: Nanolithography can be used for the creation of nanostructures and devices disclosed herein. One can use techniques, such as imprint soft writing, dip pen, photo/laser and e-beam, soft, self assembly and micro-contact lithography for the creation of nanostructures. One can create a variety of nanostructures by masking the surface with different masking techniques which produce lines of less than 10 nm, dots and other shapes followed by etching. There can be multilayer, metal, mask, metal masks, etc and different masks etched with different selective etchants e.g., acid for one and base for the other and so on.
Unusually long nanowires: One can create nanowires of incredible length by selectively masking a desired area by nanolithography and etching unprotected metallized plastic film or by making an area nonplatable/metallizable with oil followed by metallization.
Linearity: Ideal sensors are designed to have linear performance. The output signal of such a sensor is linearly proportional to the value of the measured property with parameters, such as time, concentration and total exposure. The sensitivity is defined as the ratio between output signal and measured property. The change in property is usually not linear with the size of the nanostructures. The performance of the proposed sensors/dosimeters/indicators based on nanostructures will be mainly nonlinear because they undergo an abrupt change in property. However, the performance can be made linear by having a broader distribution of the nanostructures and hence the disappearance of nanostructures can be linear. Thinner, shorter or smaller nanostructures will disappear first; followed by the next large and so on till the largest one disappears.
Disposable ChemFET: ChemFET, or chemical field-effect transistor, is a type of a field effect transistor acting as a chemical sensor. It is a structural analog of a MOSFET transistor, where the charge on the gate electrode is applied by a chemical process. It may be used to detect atoms, molecules, and ions in liquids and gases. If the materials used to make ChemFET and MOSFET are susceptible (destroyable) by an analyte, the transistors will be destroyed and one can determine the total exposure to an analyte.
Nanowave guide: Optical fiber having very thin coating of a metal, which can be any other destroyable indicating material can be used as a nanowave guide for monitoring the total exposure to an agent/analyte. The nanowave guide will be similar to that described in our patent application Ser. No. 12/478,232 for a thin metal as an indicator.
Preferred nanostructures: Though most of the nanostructures can be used for the proposed applications, the most preferred nanostructures are nanofilms, nanowires/rods and nanodots.
Metal Oxide Sensors: Gas sensing by semiconducting metal oxides is possible because changes in the electrical conductivity of oxide result from catalytic re-dox reactions at oxides' surfaces. If the semiconducting metal oxide or other materials are reactive to analytes, they will undergo an irreversible change in conductivity and can be used as the dosimeter. Reactions can be controlled by electronic structure, chemical composition, and crystal structure.
Nanoredox system: Nanoredox system is a system of an oxidation/reduction material which can be oxidized or reduced by an analyte.
Type of sensor/detector: A large number of sensors and detectors can be made from destructible nanostructures which include but are not limited to: acoustic, breathalyzer, bubble chamber, capacitance probe, charge-coupled device, chemical, chemical field-effect transistor, cloud chamber, colorimeter, density, electric current, electric potential, electrolyte, electronic nose, electro-optical, Emiconductor, fiber optics, force, galvanometer, Geiger counter, hall effect, hall probe, infrared, imaging, inductive, insulator, ionizing radiation, ion-selective electrode, leaf electroscope, magnetic, magnetic anomaly detector, magnetometer, metal detector, microwave, multimeter, neutron detection, Nichols radiometer, nondispersive infrared, ohmmeter, optical, optode, particle detector, photodiode, photoelectric, photoionization detector, photomultiplier, photomultiplier tubes, photoresistor, photoswitch, phototransistor, phototube, potentiometric, pressure, proximity, radio, redox electrode, scintillation counter, scintillation, scintillometer, subatomic particles, thermal, voltmeter and wavefront.
Advantages: The dosimeters, indicators, detectors, monitors and alike proposed here will be easy to make, simple, highly sensitive, accurate, disposable, archiveable and inexpensive.
Uniqueness: Two dimensional nanostructures (nanofilm) become sub-nano and are broken to nanoparticles and then go to atomic level before being completely converted to another chemical.

### Examples

The following examples are illustrative of carrying out the claimed inventions but should not be construed as being limitations on the scope or spirit of the instant inventions.

### Example 1

### Making of Capacitor by Coating Halocompounds

A metallized plastic film (about 3 nm thick layer of aluminum on 2 mil polyester film) was coated with solution of 15 g polyvinyl acetate in 25 g of ethyltrichloroacetate. The coating was laminated with another piece of metallized polyester film. The capacitance of the sandwich was 16.4 micro Faraday. The capacitor was radiated with 400 rads of 100 KeV X-ray. The capacitance changed to 6.1 nano faraday and after about 2 hours the metallized films became clear.

### Example 2

### Change in Electrical Resistance with Ionizing Radiation

A metallized plastic film (about 10 nm thick layer of aluminum on 4 mil polyester film) was coated with solution of 15 g polyvinyl acetate in 25 g of ethyltrichloroacetate using #3 gap bar. The coating was laminated with cellophane film. The assembly was connected to an electrometer/multimeter. The film was irradiated to 254 nm 4 watt UV lamp for a minutes at 5 cm distance as shown in FIG. 25(a). The change in electrical resistance was recorded with a video camera. The resistance changed from 0.56 kilo Ohms to 21.6 mega Ohms within a few hours and the film became almost clear (see FIGS. 25(b)).

### Example 3

### Change in Electrical Resistance of TTI Device

A TTI (time-temperature indicator) device was made as per Example 6 of our U.S. patent application Ser. No. 12/478,232. The change in electrical resistance was recorded with a video camera at room temperature. The resistance changed from 4.2 Ohms to 18.4 mega Ohms after about 18 hours and the film became almost clear.

### Example 4

### Change in Electrical Resistance with Humidity

0.5 g of potassium carbonate was dissolved in 2 g water. The solution was gradually added while homogenizing in 25 g of polyvinylpyrrolidone (33 g in 100 g of isopropanol and 50 g of methyl ethyl ketone). The solution was coated on a metallized plastic film (about 9 nm thick layer of aluminum on 2 mil polyester film) and dried at 90° C. for 15 minutes. A strip of the dried film was cut sealed with a pressure sensitive tape at both the ends to prevent/minimize diffusion of humidity. The strip was connected to an electrometer. The change in electrical resistance under ambient humidity (about 30%) and temperature (25° C.) was recorded with a video camera. The resistance changed from 35.8 Ohms to 2.52 Mega Ohms within 34 minutes and the film became almost clear.

1. An indicating system which comprises
a nanostructure; and
a means to measure the change in properties of the nanostructure as it is destroyed.

2. The indicating system of claim 1 wherein the destruction is due to one or more of: melting, fusion, dissolution, swelling, drying, etching, coagulation, conversion, transformation, crystallization, formation of defects, decomposition, reaction, diffusion, complex or adduction formation, transformation, phase, reactivity, state, size, shape, nature of doping, magnetism, porosity, permeability degradation, decay, corrosion, decomposition, disintegration, deterioration, de-metallization, coalescence, adsorption, desorption, melting, crystallization, phase change, electronic or nuclear structure, magnetism, and optical properties.

3. The indicating system of claim 1 wherein the nanostructure is less 1,000 nm in at least one dimension.

4. The indicating system of claim 1 wherein the nanostructure is one or structure selected from the group of nanoantenna, nanoballs, nanobelts, nanobipods, nanocapsules, nanocluster, nanocrystals, branched nanocrystals, nanodendrimers, nanodots, nanofilms, nanofibers, nanoflakes/sheets, nanofluids, nanolayers, nanoparticles, nanorods, nanospheres, nanosprings, nanotatrapods, branched tetrapods, nanotripods, nanotubes, nanowires, plasmon, quantum dots, and quantum wells.

5. The indicating system based of claim 1 wherein the nanostructure is a reactive or unstable organic, inorganic, organo-metallic or a biological material.

6. The indicating system of claim 1 wherein the nano structure is made from a metal.

7. The indicating system of claim 6 wherein the metal is copper, zinc, magnesium, aluminum, gold, silver silicon, or their alloys.

8. The indicating system based of claim 1 wherein the nanostructure is destroyed by an analyte or activator.

9. The indicating system of claim 8 wherein the nanostructure is destroyed by an analyte.

10. The indicating system of claim 9, wherein the analyte is a chemical or biological agent.

11. The indicating system of claim 10 wherein the chemical agent is a toxic or hazardous chemical.

12. The indicating system of claim 10 where the biological agent is a virus or a bacterium.

13. The indicating system of claim 9 wherein the analyte is energy, electromagnetic radiation, pressure, or magnetism.

14. A process of measuring change in a property of a nanostructure during its destruction.

15. A process of changing the performance of an indicating nanostructure device which comprises changing a non-linear performance of the indicating device to a linear performance by increasing the size distribution of the nanostructures in the indicating system.

16. The indicating system of claim 1 which is designed for use in monitoring total exposure to organic, inorganic, organometallic and biological compounds and agents or analytes using analytical methods.

17. The indicating system of claim 1 which is designed for monitoring time, time-temperature, thaw, freeze, humidity, ionizing radiation, temperature, microwave, sterilization, chemicals, biological or chemical agents.

18. The indicating system of claim 17 wherein the sterilization is done with steam, ethylene oxide, plasma, formaldehyde, dry heat, hydrogen peroxide or peracetic acid.

19. The indicating system of claim 1 which is a radiation dosimeter.

20. The indicating system of claim 19 wherein the radiation dosimeter is a capacitor

21. The indicating system of claim 1 wherein the nano structure is an electrode.

22. The indicating system of claim 21 wherein the electrode is an organic or inorganic conductor, semiconductor or metal.

23. The indicating system of claim 22 wherein the nanostructure is protected by a coating or stabilizing material which is a precursor, activator or transparent conductor.

24. The indicating system of claim 23 wherein the precursor is a halo-compound.

25. The indicating system of claim 1 wherein the destruction is determined an analytical method.

26. The indicating system of claim 25 wherein the analytical method is an electroanalytical method.

27. The indicating system of claim 26 wherein the electroanalytical method is ellipsometry.
